(19) United States
(12) Patent Application Publication
(10) Pub. No.: US 2016/0318893 A1
(43) Pub. Date: Nov. 3, 2016

(54) STEREO-SPECIFIC SYNTHESIS OF (13R)-MANOYL OXIDE

(71) Applicants: TECHNICAL UNIVERSITY OF DENMARK, Lyngby (DK); UNIVERSITY OF COPENHAGEN, Copenhagen K (DK)

(72) Inventors: Björn Hamberger, Kastrup (DK); Eirini Pateraki, Copenhagen (DK); Birger Lindberg MØLLERø, Brønshøj (DK); Morten NØRHOLM, Hillerød (DK); Morten Thrane Nielsen, Copenhagen (DK); Johan Andersen-Ranberg, Copenhagen (DK); Carl Jörg Bohlmann, Vancouver, British Columbia (CA); Phillipp Zerbe, North Vancouver, British Columbia (CA)

(21) Appl. No.: 15/103,838

(22) PCT Filed: Dec. 19, 2014
(86) PCT No.: PCT/EP2014/078728

§ 371 Date: Jun. 10, 2016

(30) Foreign Application Priority Data
Dec. 20, 2013 (EP) 13198742.2
Dec. 20, 2013 (EP) 13198756.2

(51) Int. Cl. C07D 311/92; C12N 9/88; C12P 17/06

(52) U.S. Cl. CPC C07D 311/92; C12P 17/06; C12N 9/88; C12Y 402/03; C07B 2200/07

(57) ABSTRACT

The present invention relates to a method for manufacturing enantiomerically pure (13R)-manoyl oxide, said method comprising the steps of contacting geranylgeranyl diphosphate (GGPP) with a class II diterpene synthase to obtain labd-13-en-8,15-diol diphosphate (LPP), and then contacting the LPP with a class I diterpene synthase to obtain (13R)-manoyl oxide. The invention further relates to (13R)-manoyl oxide obtained by the method of the invention.

### FIELD OF INVENTION

The present invention relates to a method for manufacturing enantiomerically pure (13R)-manoyl oxide, said method comprising the steps of contacting geranylgeranyl diphosphate (GGPP) with a class II diterpene synthase to obtain labd-13-en-8,15-diol diphosphate (LPP), and then contacting the LPP with a class I diterpene synthase to obtain (13R)-manoyl oxide. The invention further relates to (13R)-manoyl oxide obtained by the method of the invention. The invention furthermore relates to polypeptides with diterpene synthase activity. The invention further relates to polynucleotides encoding such polypeptides. Also provided are vectors for expression of the polypeptides and host cells expressing the polypeptides. Such polypeptides may be useful in aforementioned methods.

### BACKGROUND OF INVENTION

Manoyl oxide is a compound which exhibits a number of important properties like anti-bacterial, anticancer and anti-inflammatory activities. Manoyl oxide has so far only been detected as a side product or artefact of some reactions, and was present in a racemic mixture. Manoyl oxide derivatives also present numerous attractive properties. The (13R)-manoyl oxide epimer is a putative precursor of forskolin, a labdane diterpenoid found in the root of Coleus forskohlii (family Lamiaceae) which has received much attention for its broad range of pharmacological activities. However, despite extensive studies focusing on its medicinal properties, the biosynthesis of forskolin has not yet been conclusively elucidated.

Coleus forskohlii (synonym: Plectranthus barbatus) is a perennial medicinal shrub of the mint family (Lamiaceae) indigenous to the subtropical and temperate climate zones of India and south-east Asia. The plant has been used since ancient times in Hindu and Ayurvedic traditional medicine for treating a broad range of human health disorders. The main active compound of C. forskohlii is forskolin, a heterocyclic labdane type diterpene found in the roots of the plant. The diverse pharmaceutical known and potential applications for forskolin extend from alleviation of glaucoma, anti-HIV or antitumor activities to treatment of hypertension and heart failure. The efficacy of forskolin relies on activation of the adenylate cyclase enzyme leading to a marked increase of the intracellular level of cAMP (3′-5′-cyclic adenosine monophosphate) in mammalian in vitro and in vivo systems. The semi-synthetic forskolin derivative NKH477 has been approved for commercial use in Japan for treatment of cardiac surgery complications, heart failure, and cerebral vasospasm, while a forskolin eye drop solution was recently approved as an effective treatment for glaucoma.

The chemical complexity of C. forskohlii has been well studied and a total of 68 different diterpenoids have been isolated and identified from different tissues of the plant, of which 25 belong to the class of abietanes and 43 to the class of labdanes. While the tricyclic abietane diterpenes have been reported to accumulate predominantly in the aerial parts, labdane diterpenoids with a bicyclic decalin core were detected primarily in the roots. Forskolin is a representative of an unusual series of tricyclic (8,13)-epoxy-labdanes, characteristic for this plant.

Manoyl-oxide has to this date only been detected as experimental artefact (Zerbe et al., 2012; Günnewich et al., 2013). Enzymatic conversion leading to production of manoyl oxide has at present never been reported. Thus pure enantiomers of manoyl oxide are currently not available. Methods of producing enantiomerically pure enantiomers of manoyl-oxide, including (13R)-manoyl oxide are needed. Also polypeptides capable of producing manoyl oxide are needed.

### SUMMARY OF INVENTION

In one aspect, the invention relates to a method of manufacturing (13R)-manoyl oxide, said method comprising the steps of:
(i) providing geranylgeranyl diphosphate (GGPP);
(ii) contacting GGPP of step (i) with a first polypeptide having a sequence at least 70% identical to SEQ ID NO: 1 [CfTPS2] or SEQ ID NO: 2 [SsLPPS], thus obtaining labd-13-en-8,15-diol diphosphate (LPP);
(iii) contacting the LPP of step (ii) with a second polypeptide having a sequence at least 70% identical to SEQ ID NO: 3 [CfTPS4], SEQ ID NO: 4 [CfTPS3] or SEQ ID NO: 5 [EpTPS8];
   thus obtaining (13R)-manoyl-oxide.

In another aspect, the invention relates to (13R)-manoyl oxide obtained by the method of the invention.

In another aspect, the invention relates to polypeptides having a diterpene synthase (diTPS) activity. The invention further relates to polynucleotides encoding such polypeptides and to vectors comprising such polynucleotides. The invention finally relates to a host cell comprising such vectors and/or such polynucleotides. The polypeptides of the invention are relevant for catalysing enzymatic synthesis of manoyl oxide.

Thus, in one aspect, the invention relates to polypeptides having a diterpene synthase activity and comprising:

i) an amino acid sequence selected from the group consisting of SEQ ID NO: 1 [CfTPS2], SEQ ID NO: 4 [CfTPS3], SEQ ID NO: 3 [CfTPS4] and SEQ ID NO: 5 [EpTPS8];
ii) a biologically active sequence variant of said polypeptide, wherein the sequence variant has at least 75% sequence identity to said SEQ ID NO: 1 [CfTPS2], SEQ ID NO: 4 [CfTPS3], SEQ ID NO: 3 [CfTPS4] or SEQ ID NO: 5 [EpTPS8];
iii) a biologically active fragment of at least at least 50 contiguous amino acids of any of i) through ii), said fragment having at least 75% sequence identity to SEQ ID NO: 1 [CfTPS2], SEQ ID NO: 4 [CfTPS3], SEQ ID NO: 3 [CfTPS4] or SEQ ID NO: 5 [EpTPS8] in a range of overlap of at least 50 amino acids, wherein the biological activity is diterpene synthase activity.

In a further aspect, the invention relates to a polynucleotide encoding a polypeptide of the invention.

In another aspect, the invention relates to a vector comprising at least one polynucleotide of the invention.

In yet another aspect, the invention relates to a cell comprising a polynucleotide of the invention and/or a vector of the invention.

Zerbe et al., 2013 describes gene discovery of modular diterpene metabolism in nonmodel systems. For example GrTPS1 and GrTPS6 are disclosed. These Gr enzymes are very different to the TPS enzymes disclosed herein. Thus, GrTPS1 only shares 43% sequence identity with CfTPS2, whereas GrTPS6 shares as little as 34% and 32% sequence identity with CfTPS3 and CfTPS4, respectively.

### DESCRIPTION OF DRAWINGS

FIG. 1: Localization of oil bodies within the root cork of C. forskohlii. (A) Cross section of entire root with thick fissured cork. Lower right inset: the location of cork cells. (B) Rows of cork cells of different intensity, each with one prominent oil body. (C-F) Confocal imaging of Nile Red labeled oil bodies. (C) Cell with two oil bodies (same motif as D-F) seen in transmission channel. Discrimination between neutral lipids seen as bright deposits (D) and polar lipids seen in the magenta spectrum (E), also shown as overlay (F). The bar represents 200 μm (A) and 10 μm (B-F).

FIG. 2: Forskolin content (mg g⁻¹DW) as determined by HPLC-ELSD analysis from different tissues of C. forskohlii. (Ck), root cork; (CS), root stele and cortex; (FI), flowers; (St) stems and (Lv) leaves. Data are the mean±SE of three independent biological replicates. The Y axis shows the forskolin content (mg/g dry weight).

FIG. 3: Selected diterpenes detected in C. forskohlii oil bodies. FS: forskolin standard; RC: root cork; IOL: isolated oil bodies. (A) LC-MS analysis of forskolin (410 [forskolin]+23 [Na+]) in isolated oil bodies and in root cork tissue from C. forskohlii. The X axis shows the time in minutes. (B) GC-MS analysis of manoyl oxide in isolated oil bodies and in root cork tissue from C. forskohlii. The X axis shows the time in minutes. (C) Bright field microscope image of isolated C. forskohlii oil bodies. The bar represents 5 μm. (D) Molecular structure of (13R)-manoyl oxide. (E) Mass spectrum obtained from manoyl oxide identified in root cork tissue (top) and reference spectrum (bottom) from Wiley mass spectrum database.

FIG. 4: Phylogenetic classification of C. forskohlii diterpene synthases with known class II (A) and class I (B) sequences. The phylograms are rooted with the bifunctional ent-copalyl diphosphate synthase/ent-kaurene synthase from the moss Physcomitrella patens. Asterisks indicate nodes supported by >80% bootstrap confidence and the scale bar indicates 0.1 amino acid changes. The numbers indicated at each enzyme are referred to the enzymatic products, the structures of which are given on the right.

FIG. 5: Relative expression of CfTPS genes in C. forskohlii tissues. (Ck), root cork; (CS), root stele and cortex; (FI), flowers; (St), stems and (Lv), leaves. Transcript abundance of CfTPS genes expressed in arbitrary units was measured by qPCR using the translation initiation factor (TIF4a) for normalization. Each value represents the average of three biological replicates, each of which was performed in at least three technical replicates.

FIG. 6: GC-MS analysis of in vitro assays with C. forskohlii diTPS. IS, internal standard (1 ppm 1-eicosene). (A) In vitro assays with CfTPS2 alone and coupled assays with CfTPS2 and CfTPS3 and CfTPS4. Extracts of CfTPS2 assays were treated with calf intestinal alkaline phosphatase (CIP). The X axis shows the retention time (minutes). (B) In vitro assays with CfTPS1 and coupled with CfTPS3 and CfTPS4. Extracts of CfTPS1 were treated with CIP. The X axis shows the retention time (minutes). (a), (13R)-manoyl oxide; (b), (13S)-manoyl oxide; (g), labd-13-en-8,15-diol and (f), labden-8-ol; (d), miltiradiene and (h), copal-15-ol. (C) Mass spectra of compounds identified from assays. Structures tentatively identified as described in Materials and Methods.

FIG. 7: GC-MS analysis of hexane extracts from N. benthamiana transiently expressing C. forskohlii diTPS. (A) Extracted ion chromatogram of EIC: 275 m/z. (a), (13R)-manoyl oxide and (b), (13S)-manoyl oxide. (B) Extracted ion chromatogram of EIC: 272 m/z. (c), dehydroabietadiene; (d), miltiradiene and trace amount of (e), abietadiene.

FIG. 8: Scheme of the biosynthetic routes from GGPP to specialized and general diterpenoids of the abietane, labdane and ent-kaurene class. Dashed arrows indicate reactions without experimental evidence in C. forskohlii. ¹Detection of (+)-ferruginol in C. forskohlii was reported earlier (Kelecom, 1983); ²CYP76AH1 from the close relative Salvia miltiorrhiza was shown to convert miltiradiene to ferruginol (Guo et al., 2013). A: universal precursor; B: diphosphate intermediates; C: diterpene backbone; D: representative functional diterpenoids.

FIG. 9: Clustal alignment of the class II diTPS. The cDNA sequences encoding CfTPS2 and SsLPPS were aligned using the Clustal omega from the EMBL-EBI (http://www.ebi.ac.uk/Tools/msa/clustalo/help/).

FIG. 10: Clustal alignment of the class I diTPS. The cDNA sequences encoding CfTPS3, CfTPS4 and EpTPS8 were aligned using the Clustal omega from the EMBL-EBI (http://www.ebi.ac.uk/Tools/msa/clustalo/help/).

### DETAILED DESCRIPTION OF THE INVENTION

In one aspect, the invention relates to a method of manufacturing (13R)-manoyl oxide, said method comprising the steps of:
(i) providing geranylgeranyl diphosphate (GGPP);
(ii) contacting GGPP of step (i) with a first polypeptide having the sequence of SEQ ID NO:1 [CfTPS2], or SEQ ID NO: 2 [SsLPPS] or a biologically active sequence variant of said polypeptide, wherein the sequence variant has at least 75% sequence identity to SEQ ID NO: 1 [CfTPS2] or SEQ ID NO: 2 [SsLPPS], thus obtaining labd-13-en-8,15-diol diphosphate (LPP);
(iii) contacting the LPP of step (ii) with a second polypeptide having the sequence of SEQ ID NO: 3 [CfTPS4], SEQ ID NO: 4 [CfTPS3] or SEQ ID NO: 5 [EpTPS8] or a biologically active sequence variant of said polypeptide, wherein the sequence variant has at least 75% sequence identity to SEQ ID NO: 3 [CfTPS4], SEQ ID NO: 4 [CfTPS3] or SEQ ID NO: 5 [EpTPS8];
   thus obtaining (13R)-manoyl-oxide.

In another aspect, the invention relates to (13R)-manoyl oxide obtained by the method of the invention.

In one aspect the invention relates to polypeptides having a diterpene synthase (diTPS) activity. The invention further relates to polynucleotides encoding such polypeptides and to vectors comprising such polynucleotides. The invention finally relates to a host cell comprising such vectors and/or such polynucleotides. The polypeptides of the invention are relevant for catalysing enzymatic synthesis of manoyl oxide.

In one aspect, the invention relates to polypeptides having a diterpene synthase activity and comprising:

i) an amino acid sequence selected from the group consisting of SEQ ID NO: 1 [CfTPS2], SEQ ID NO: 4 [CfTPS3], SEQ ID NO: 3 [CfTPS4] and SEQ ID NO: 5 [EpTPS8];
ii) a biologically active sequence variant of said polypeptide, wherein the sequence variant has at least 75% sequence identity to said SEQ ID NO: 1 [CfTPS2], SEQ ID NO: 4 [CfTPS3], SEQ ID NO: 3 [CfTPS4] or SEQ ID NO: 5 [EpTPS8];
iii) a biologically active fragment of at least at least 50 contiguous amino acids of any of i) through ii), said fragment having at least 75% sequence identity to SEQ ID NO: 1 [CfTPS2], SEQ ID NO: 4 [CfTPS3], SEQ ID NO: 3 [CfTPS4] or SEQ ID NO: 5 [EpTPS8] in a range of overlap of at least 50 amino acids, wherein the biological activity is diterpene synthase activity.

In a further aspect, the invention relates to a polynucleotide encoding a polypeptide of the invention.

In another aspect, the invention relates to a vector comprising at least one polynucleotide of the invention.

In yet another aspect, the invention relates to a cell comprising a polynucleotide of the invention and/or a vector of the invention.

### DEFINITIONS

Amino acid: Any synthetic or naturally occurring amino carboxylic acid, including any amino acid occurring in peptides and polypeptides including proteins and enzymes synthesized in vivo thus including modifications of the amino acids. The term amino acid is herein used synonymously with the term “amino acid residue” which is meant to encompass amino acids as stated which have been reacted with at least one other species, such as 2, for example 3, such as more than 3 other species. The generic term amino acid comprises both natural and non-natural amino acids any of which may be in the “D” or “L” isomeric form.

Diterpene synthases (diTPS): Diterpene synthases (diTPS, EC 4.2.3.X) are enzymes capable of synthesising diterpene olefins and alcohols by sequential cycloisomerisation of the substrate geranylgeranyl-diphosphate (GGPP). DiTPS can be sorted in two classes, depending on the presence of a conserved motif. Class I diTPS contain an active site with a DDxxD motif, where D is an aspartic acid and x is any amino acid. Class II diTPS contain an active site with a DxDD motif, where D is an aspartic acid and x is any amino acid. Bifunctional classI/II diTPS contain two active sites, with a DDxxD and a DxDD motif, respectively.

Diteroenoid: As used herein, a diterpenoid is an unsaturated hydrocarbon based on the isoprene unit (C₅H₈), and having a general formula C_{5X}H_{8X}. A diterpene contains a backbone of 20 carbon atoms, which can be decorated by additional groups, e.g. by esterification. A diterpenoid also is a type of diterpene. A diterpenoid can derive from geranylgeranyl pyrophosphate (GGPP). Diterpenoids include all types of molecules derived from GGPP with a very broad range of functionalization. Examples of diterpenoids are olefins and diterpene alcohols.

Enantiomer: An enantiomer or enantiomorph or epimer is one of two stereoisomers that are mirror images of each other that are non-superposable. In other words, an enantiomer is a chiral molecule having a non-superposable mirror image. Enantiomers have, when present in a symmetric environment, identical chemical and physical properties except for their ability to rotate plane-polarized light (+/−) by equal amounts but in opposite directions. Enantiomers of one compound often react differently with other substances that are also enantiomers. Since many molecules in the living organisms are enantiomers themselves, there is sometimes a marked difference in the effects of two enantiomers on these organisms. In drugs, for example, often only one of a drug's enantiomers is responsible for the desired physiologic effects, while the other enantiomer is less active, inactive, or sometimes even responsible for adverse effects. Thus drugs composed of only one enantiomer (enantiomerically pure) can be developed to enhance the pharmacological efficacy and possibly dampen some side effects.

Enantiomerically pure: Enantiomerically pure, or enantiopure, refers to samples having, within the limits of detection, molecules of only one chirality.

Fragment: is used to indicate a non-full length part of a polynucleotide or polypeptide. Thus, a fragment is itself also a polynucleotide or polypeptide, respectively.

Identity: The determination of percent identity between sequences such as polynucleotide sequences or amino acid sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN and BLASTP programs of Altschul, et al. (1990) J. Mol. Biol. 215:403-410. In order to characterize the identity, subject sequences are aligned so that the highest order homology (match) is obtained. Based on these general principles, the “percent identity” of two polynucleotide sequences may be determined using the BLASTN algorithm [Tatiana A. Tatusova, Thomas L. Madden: Blast 2 sequences—a new tool for comparing protein and nucleotide sequences; FEMS Microbiol. Lett. 1999 174 247-250], which is available from the National Center for Biotechnology Information (NCBI) web site (http://www.ncbi.nlm.nih.gov), and using the default settings suggested here (i.e. Reward for a match=1; Penalty for a mismatch=−2; Strand option=both strands; Open gap=5; Extension gap=2; Penalties gap x_dropoff=50; Expect=10; Word size=11; Filter on). The BLASTN algorithm determines the % sequence identity in a range of overlap between two aligned nucleotide sequences. Another preferred, non-limiting example of a mathematical algorithm utilized for the comparison of sequences is the CLUSTAL W (1.7) alignment algorithm (Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680.). CLUSTAL W can be used for multiple sequence alignment preferably using BLOSUM 62 as scoring matrix. When calculating sequence identities, CLUSTAL W includes any gaps made by the alignment in the length of the reference sequence. Sequence identities are calculated by dividing the number of matches by the length of the aligned sequences with gaps. In general, the sequence identity is calculated with reference to the entire length of the reference sequence. Thus, a candidate sequence sharing 80% amino acid identity with a reference sequence, requires that, following alignment, 80% of the amino acids in the candidate sequence are identical to the corresponding amino acids in the reference sequence.

Operative: An operative domain in relation to a class I or class II domain refers to a domain securing the biological processing of the polypeptide.

Plastidial targeting signal: A short sequence of amino acids that determines that a polypeptide should locate to the plastid in a plant cell.

Polynucleotide: A chain or sequence of nucleotides that convey genetic information. In regards to the present invention the polynucleotide is a deoxyribonucleic acid (DNA).

Polypeptide: Plurality of covalently linked amino acid residues defining a sequence and linked by amide bonds. The term is used analogously with oligopeptide and peptide. The natural and/or non-natural amino acids may be linked by peptide bonds or by non-peptide bonds. The term peptide also embraces post-translational modifications introduced by chemical or enzyme-catalyzed reactions, as are known in the art. The term can refer to a variant or fragment of a polypeptide.

Promoter: A binding site in a DNA chain at which RNA polymerase binds to initiate transcription of messenger RNA by one or more nearby structural genes. An inducible promoter refers to a promoter where initiation of transcription can be induced by e.g. addition of a compound to the growth medium or by changing the temperature.

Racemic mixture: A racemic mixture contains equal parts of an optically active isomer and its enantiomer and has zero net rotation of plane-polarized light.

Substantially pure: As used herein, substantially pure means sufficiently homogeneous to appear free of readily detectable impurities as determined by standard methods of analysis, such as thin layer chromatography (TLC), gel electrophoresis and high performance liquid chromatography (HPLC), gas-chromatography mass-spectrometry (GC-MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound can, however, be a mixture of stereoisomers or isomers. In such instances, further purification might increase the specific activity of the compound.

Transient expression: Transient expression refers to temporary expression of a polypeptide, for a limited period of time. Transient expression can be controlled by inducible and repressible promoters, agroinfiltration of plant cells with a bacterium such as Agrobacterium tumefaciens, and other methods known in the art.

Variant: a ‘variant’ of a given reference polynucleotide or polypeptide refers to a polynucleotide or polypeptide that displays a certain degree of sequence homology/identity to said reference polynucleotide or polypeptide but is not identical to said reference polynucleotide or polypeptide.

Vector: A vector is a DNA molecule or an organism comprising a DNA molecule used as a vehicle to artificially carry foreign genetic material into another cell, where the DNA molecule can be replicated and/or expressed. The vectors herein may be plasmids, viral vectors, cosmids, bacterial vectors and artificial chromosomes.

Method for Manufacturing Enantiomerically Pure (13R)-manoyl-oxide

In one aspect, the invention relates to a method of manufacturing substantially pure (13R)-manoyl oxide, said method comprising the steps of:
(i) providing geranylgeranyl diphosphate (GGPP);
(ii) contacting GGPP of step (i) with a first polypeptide having a sequence at least 70% identical to SEQ ID NO: 1 [CfTPS2] or SEQ ID NO: 2 [SsLPPS], thus obtaining labd-13-en-8,15-diol diphosphate (LPP);
(iii) contacting the LPP of step (ii) with a second polypeptide having a sequence at least 70% identical to SEQ ID NO: 3 [CfTPS4], SEQ ID NO: 4 [CfTPS3] or SEQ ID NO: 5 [EpTPS8];
   thus obtaining (13R)-manoyl-oxide.

Manoyl oxide is a compound of general formula C₂₀H₃₄O. It has been detected as an experimental artefact in a racemic mixture of the (13R) epimer (I) and (13S) epimer (II).

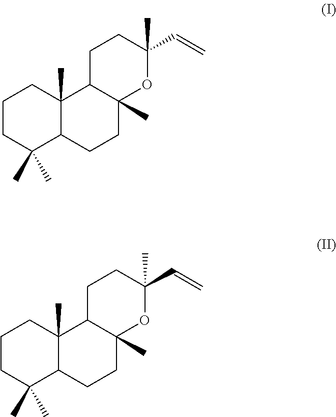

(13R)-manoyl oxide has been hypothesized to be a precursor for the synthesis of forskolin.

The inventors have found that (13R)-manoyl oxide may be synthesised stereospecifically, i.e. in an enantiomerically substantially pure form, by the following reaction:

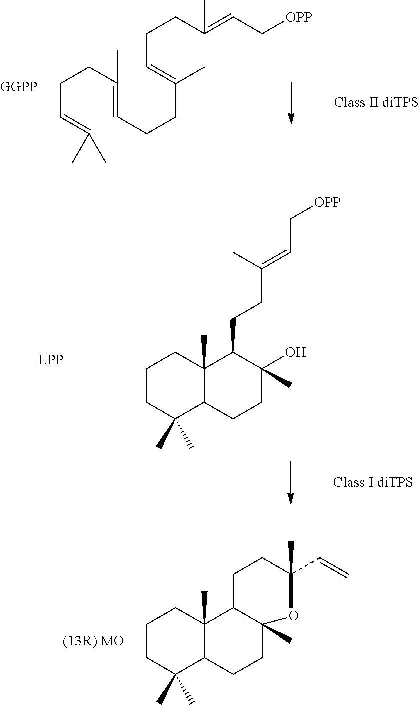

where GGPP is geranylgeranyl diphosphate, LPP is labd-13-en-8,15-diol diphosphate, and (13R) MO is (13R)-manoyl oxide.

Racemic mixtures of manoyl oxide have been disclosed, however it is very difficult to separate an enantiomerically pure (13R)-MO from such a racemic mixture. Thus, currently no established methods for purifying large amounts (e.g. gram scales) of enantiomerically pure (13R)-manoyl oxide are available.

The first step of the reaction is catalysed by a diterpene synthase (diTPS) having a class II diTPS activity. The class II diTPS catalyses protonation-initiated cationic cycloisomerization of GGPP to LPP. The reaction is terminated either by deprotonation or by water capture of the diphosphate carbocation. The first step of the reaction may be catalysed by any of the Class II diTPS described herein below in the section “Class II diterpene synthase”. Class II diTPS particularly relevant for the invention are TPS2 from Coleus forskohlii (CfTPS2) and LPPS from Salvia sclarea (SsLPPS).

The method of the invention allows manufacturing of (13R)-manoyl oxide. In some embodiments, the (13R)-manoyl oxide obtained is substantially pure. Thus in some embodiments, the (13R)-manoyl oxide is more than 90% pure, such as 91% pure, such as 92% pure, such as 93% pure, such as 94% pure, such as 95% pure, such as 96% pure, such as 97% pure, such as 98% pure, such as 99% pure, such as 100% pure. In preferred embodiments, the (13R)-manoyl oxide manufactured by the method of the invention is more than 95% pure. In a preferred embodiment, the (13R)-manoyl oxide is 99% pure. In another preferred embodiment, the (13R)-manoyl oxide is 100% pure.

Also disclosed herein is a method for manufacturing (13R)-manoyl oxide which is enantiomerically pure. The manoyl-oxide manufactured by the method of the invention is essentially (13R)-manoyl oxide. In some embodiments, the (13R)-manoyl oxide is more than 90% enantiomerically pure, such as 91% enantiomerically pure, such as 92% enantiomerically pure, such as 93% enantiomerically pure, such as 94% enantiomerically pure, such as 95% enantiomerically pure, such as 96% enantiomerically pure, such as 97% enantiomerically pure, such as 98% enantiomerically pure, such as 99% enantiomerically pure, such as 100% enantiomerically pure. In preferred embodiments, the (13R)-manoyl oxide manufactured by the method of the invention is more than 95% enantiomerically pure. In a preferred embodiment, the (13R)-manoyl oxide is 99% enantiomerically pure. In another preferred embodiment, the (13R)-manoyl oxide is 100% enantiomerically pure.

The product obtained by performing the method of the invention is essentially free of (13S)-manoyl oxide. In some embodiments, the product obtained comprises less than 10% (13S)-manoyl oxide, such as less than 9% (13S)-manoyl oxide, such as less than 8% (13S)-manoyl oxide, such as less than 7% (13S)-manoyl oxide, such as less than 6% (13S)-manoyl oxide, such as less than 5% (13S)-manoyl oxide, such as less than 4% (13S)-manoyl oxide, such as less than 3% (13S)-manoyl oxide, such as less than 2% (13S)-manoyl oxide, such as less than 1%(13S)-manoyl oxide, such as 0% (13S)-manoyl oxide. In a preferred embodiment, the product obtained comprises less than 1% (13S)-manoyl oxide. In another preferred embodiment, the product obtained comprises no (13S)-manoyl oxide.

The method of the invention is performed by contacting GGPP with a first polypeptide having a class II diTPS activity and a second polypeptide having a class I diTPS activity.

In some embodiments, the first polypeptide has a sequence at least 70% identical to, such as at least 75% identical to, such as at least 80% identical to, such as at least 85% identical to, such as at least 90% identical to, such as at least 95% identical to, such as 100% identical to SEQ ID NO: 1 [CfTPS2].

In other embodiments, the first polypeptide has a sequence at least 70% identical to, such as at least 75% identical to, such as at least 80% identical to, such as at least 85% identical to, such as at least 90% identical to, such as at least 95% identical to, such as 100% identical to SEQ ID NO: 2 [SsLPPS].

The second step of the reaction is catalysed by a diTPS having a class I diTPS activity. It catalyzes cleavage of the diphosphate group of LPP and additional cyclization or rearrangement reactions on the resulting carbocation, yielding (13R)-manoyl oxide. As with the class II diTPSs, deprotonation or water capture terminate the class I diTPS reaction. The second step of the reaction may be catalysed by any of the Class I diTPS described herein below in the section “Class I diterpene synthase”. Class I diTPS particularly relevant for the invention are TSP3 and TPS4 from Coleus forskohlii (CfTPS3 and CfTPS4, respectively) and TPS8 from Euphorbia peplus (EpTPS8).

In some embodiments, the second polypeptide has a sequence at least 70% identical to, such as 75% identical to, such as at least 80% identical to, such as at least 85% identical to, such as at least 90% identical to, such as at least 95% identical to, such as 100% identical to SEQ ID NO: 3 [CfTPS4].

In other embodiments, the second polypeptide has a sequence at least 70% identical to, such as at least 75% identical to, such as at least 80% identical to, such as at least 85% identical to, such as at least 90% identical to, such as at least 95% identical to, such as 100% identical to SEQ ID NO: 4 [CfTPS3].

In other embodiments, the second polypeptide has a sequence at least 70% identical to, such as at least 75% identical to, such as at least 80% identical to, such as at least 85% identical to, such as at least 90% identical to, such as at least 95% identical to, such as 100% identical to SEQ ID NO: 5 [EpTPS8].

In a preferred embodiment, the first polypeptide has a sequence at least 70% identical to, such as at least 75% identical to, such as at least 80% identical to, such as at least 85% identical to, such as at least 90% identical to, such as at least 95% identical to, such as 100% identical to SEQ ID NO: 1 [CfTPS2] and the second polypeptide has a sequence at least 75% identical to, such as at least 80% identical to, such as at least 85% identical to, such as at least 90% identical to, such as at least 95% identical to, such as 100% identical to SEQ ID NO: 3 [CfTPS4]. In one embodiment, the first polypeptide has a sequence identical to SEQ ID NO: 1 [CfTPS2] and the second polypeptide has a sequence identical to SEQ ID NO: 3 [CfTPS4].

For example, the first polypeptide may be a biologically active sequence variant of CfTPS2 of SEQ ID NO:1, wherein the sequence variant is at least 75% identical to, such as at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 1 [CfTPS2], and the second polypeptide may be a biologically active sequence variant of CfTPS4 of SEQ ID NO:3, wherein the sequence variant is at least 75% identical to, such as at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 3 [CfTPS4].

In a preferred embodiment, the first polypeptide has a sequence at least 70% identical to, such as at least 75% identical to, such as at least 80% identical to, such as at least 85% identical to, such as at least 90% identical to, such as at least 95% identical to, such as 100% identical to SEQ ID NO: 1 [CfTPS2] and the second polypeptide has a sequence at least 75% identical to, such as at least 80% identical to, such as at least 85% identical to, such as at least 90% identical to, such as at least 95% identical to, such as 100% identical to SEQ ID NO: 4 [CfTPS3]. In one embodiment, the first polypeptide has a sequence identical to SEQ ID NO: 1 [CfTPS2] and the second polypeptide has a sequence identical to SEQ ID NO: 4 [CfTPS3].

Thus it is preferred that the first polypeptide may be a biologically active sequence variant of CfTPS2 of SEQ ID NO:1, wherein the sequence variant is at least 75% identical to, such as at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 1 [CfTPS2], and the second polypeptide may be a biologically active sequence variant of CfTPS3 of SEQ ID NO:4, wherein the sequence variant is at least 75% identical to, such as at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 4 [CfTPS3]. In particular, the first polypeptide may be CfTPS2 of SEQ ID NO:1 or a biologically active sequence variant of CfTPS2 of SEQ ID NO:1, wherein the sequence variant is at least 80% identical to, EQ ID NO: 1 [CfTPS2], and the second polypeptide may be CfTPS3 of SEQ ID NO:4 or a biologically active sequence variant of CfTPS3 of SEQ ID NO:4, wherein the sequence variant is at least 80% identical to SEQ ID NO: 4 [CfTPS3]. This may in particular be preferred in embodiments of the invention relating to methods for producing (13R)-manoyl oxide that is more than 95% enantiomerically pure.

In another embodiment, the first polypeptide has a sequence at least 70% identical to, such as at least 75% identical to, such as at least 80% identical to, such as at least 85% identical to, such as at least 90% identical to, such as at least 95% identical to, such as 100% identical to SEQ ID NO: 1 [CfTPS2] and the second polypeptide has a sequence at least 75% identical to, such as at least 80% identical to, such as at least 85% identical to, such as at least 90% identical to, such as at least 95% identical to, such as 100% identical to SEQ ID NO: 5 [EpTPS8]. In one embodiment, the first polypeptide has a sequence identical to SEQ ID NO: 1 [CfTPS2] and the second polypeptide has a sequence identical to SEQ ID NO: 5 [EpTPS8].

In another embodiment, the first polypeptide has a sequence at least 70% identical to, such as at least 75% identical to, such as at least 80% identical to, such as at least 85% identical to, such as at least 90% identical to, such as at least 95% identical to, such as 100% identical to SEQ ID NO: 2 [SsLPPS] and the second polypeptide has a sequence at least 75% identical to, such as at least 80% identical to, such as at least 85% identical to, such as at least 90% identical to, such as at least 95% identical to, such as 100% identical to SEQ ID NO: 3 [CfTPS4]. In one embodiment, the first polypeptide has a sequence identical to SEQ ID NO: 2 [SsLPPS] and the second polypeptide has a sequence identical to SEQ ID NO: 3 [CfTPS4].

In another embodiment, the first polypeptide has a sequence at least 70% identical to, such as at least 75% identical to, such as at least 80% identical to, such as at least 85% identical to, such as at least 90% identical to, such as at least 95% identical to, such as 100% identical to SEQ ID NO: 2 [SsLPPS] and the second polypeptide has a sequence at least 75% identical to, such as at least 80% identical to, such as at least 85% identical to, such as at least 90% identical to, such as at least 95% identical to, such as 100% identical to SEQ ID NO: 4 [CfTPS3]. In one embodiment, the first polypeptide has a sequence identical to SEQ ID NO: 2 [SsLPPS] and the second polypeptide has a sequence identical to SEQ ID NO: 4 [CfTPS3].

In another embodiment, the first polypeptide has a sequence at least 70% identical to, such as at least 75% identical to, such as at least 80% identical to, such as at least 85% identical to, such as at least 90% identical to, such as at least 95% identical to, such as 100% identical to SEQ ID NO: 2 [SsLPPS] and the second polypeptide has a sequence at least 75% identical to, such as at least 80% identical to, such as at least 85% identical to, such as at least 90% identical to, such as at least 95% identical to, such as 100% identical to SEQ ID NO: 5 [EpTPS8]. In one embodiment, the first polypeptide has a sequence identical to SEQ ID NO: 2 [SsLPPS] and the second polypeptide has a sequence identical to SEQ ID NO: 5 [EpTPS8].

In order to obtain enantiomerically pure (13R)-manoyl oxide it is preferred that the first and the second polypeptides are present at a stoichiometry that allows enantiomerically pure (13R)-manoyl oxide to be yielded by the reaction. The first and second polypeptides are preferably present in the reaction at a stoichiometry ratio between 2:1 and 1:2. Preferably, the first and the second polypeptides are present in equal amounts, i.e. in a stoichiometry 1.1. Other stoichiometry ratios may lead to unbalanced reactions, where the produced manoyl oxide is in a racemic mixture, where manoyl-oxide is present both in the form of (13R)-manoyl oxide and (13S)-manoyl oxide.

In some embodiments, the method of the invention further comprises a step of recovering (13R)-manoyl oxide by methods known in the art.

The method of the invention can be performed in vivo. The first and the second polypeptides may be heterologously expressed in a host organism by methods known in the art. The host organism may be a prokaryote or a eukaryote. In some embodiments, the host organism is selected from the group comprising bacteria, yeast, fungi, plants, insects and mammals. The host organism may be selected from the group comprising Escherichia coli, Saccharomyces cerevisiae, Schizosaccharomyces pombe, Nicotiana benthamiana and Physcomitrella patens. Thus in one embodiment, the first and the second polypeptides are heterologously expressed in Escherichia coli. In another embodiment, the first and the second polypeptides are heterologously expressed in Saccharomyces cerevisiae. Thus in yet another embodiment, the first and the second polypeptides are heterologously expressed in Nicotiana benthamiana. In a preferred embodiment, the first and the second polypeptides are heterologously expressed from Nicotiana benthamiana. Methods for expressing the first and the second polypeptide in a host organism are known to those skilled in the art.

For performing the method of the invention in vivo, the GGPP may be provided in a composition or it may be provided by the host organism or by a second host organism.

In some embodiments, the host organism is capable of secreting the first polypeptide and the second polypeptide. The GGPP is provided in a composition or is provided by the host organism or by a second host organism, capable of secreting the GGPP. Thus in some embodiments, the reaction occurs in a composition comprising the first and the second polypeptides secreted by the host organism and GGPP provided in the composition or secreted by the host organism or a second host organism. The second host organism may be selected from the group comprising Escherichia coli, Saccharomyces cerevisiae, Schizosaccharomyces pombe, Nicotiana benthamiana and Physcomitrella patens. Preferably, the second host organism is identical to the host organism capable of heterologously expressing the first and the second polypeptides.

The method of the invention can also be performed in a host cell. The first and the second polypeptides may be heterologously expressed in the host cell by methods known in the art. The host cell may be a prokaryote or a eukaryote. In some embodiments, the host cell is selected from the group comprising bacteria, yeast, fungi, plants, insects and mammals. The host cell may be selected from the group comprising Escherichia coli, Saccharomyces cerevisiae, Schizosaccharomyces pombe, Nicotiana benthamiana and Physcomitrella patens. Thus in one embodiment, the first and the second polypeptides are heterologously expressed in Escherichia coli. In another embodiment, the first and the second polypeptides are heterologously expressed in Saccharomyces cerevisiae. In yet another embodiment, the first and the second polypeptides are heterologously expressed in Nicotiana benthamiana. In a preferred embodiment, the first and the second polypeptides are heterologously expressed from Nicotiana benthamiana. Methods for expressing the first and the second polypeptide in a host organism are known to those skilled in the art.

The starting substrate of the reaction being GGPP, it may be an advantage that the host cell is further capable of producing GGPP. Thus the host cell may be genetically engineered to be capable of synthesising GGPP. This can be performed by methods known in the art. In a preferred embodiment, the GGPP is produced via the plastidial methylerythritol 4-phosphate (MEP) pathway, which can be cloned in the host cell.

In other embodiments, the method of manufacturing (13R)-manoyl oxide is performed in vitro. In such embodiments, the first and the second polypeptides may be heterologously expressed from a host organism as described above, and subsequently purified before being contacted with GGPP provided in a composition. The GGPP may itself be produced by a host organism as detailed above.

The method of the invention may in some embodiments comprise a step of recovering the (13R)-manoyl oxide by methods known in the art. Such methods may involve solid-phase microextraction from plant leaves when the method is performed in a plant. (see for example Spanner et al., 2013). The host cell may also be capable of secreting (13R)-manoyl oxide, thereby facilitating its recovery.

The invention further relates to a non-enzymatic method for manufacturing forskolin using (13R)-manoyl oxide substrate. In some embodiments, (13R)-manoyl oxide is converted via chemical reactions performed in vitro to produce forskolin. Such chemical reactions do not comprise enzymatic reactions. These non-enzymatic reactions are well known by those of skill in the art.

Class I Diterpene Synthases

In one aspect the invention provides a polypeptide having diPTS activity. Said polypeptide is useful in the methods of manufacturing (13R)-manoyl oxide according to the invention,

The polypeptide of the invention has a diTPS activity. In some embodiments, the polypeptide has a class I diTPS activity. Class I diTPS are capable of cleaving diphosphate groups and performing rearrangement reactions such as cyclization.

Some polypeptides of the invention have a class I diTPS activity and catalyse cleavage of the diphosphate group of LPP and additional cyclization or rearrangement reactions on the resulting carbocation, yielding (13R)-manoyl oxide. Deprotonation or water capture terminate the class I diTPS reaction. Class I diTPS relevant for the invention are TSP3 and TPS4 from Coleus forskohlii (CfTPS3 and CfTPS4, respectively) and TPS8 from Euphorbia peplus (EpTPS8).

In one embodiment the polypeptide of the invention comprises:
i) an amino acid sequence identical to SEQ ID NO: 4 [CfTPS3],
ii) a biologically active sequence variant of said polypeptide, wherein the sequence variant is at least 75% identical to, such as at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 4 [CfTPS3],
iii) a biologically active fragment of at least 50 contiguous amino acids of any of i) through ii), said fragment having at least 75% sequence identity to SEQ ID NO: 4 [CfTPS3] in a range of overlap of at least 50 amino acids, wherein the biological activity is diterpene synthase activity, preferably class I diterpene synthase activity.

In a preferred embodiment, the polypeptide comprises a biologically active sequence variant having an amino acid sequence at least 95% identical to SEQ ID NO: 4 [CfTPS3]. In another preferred embodiment, the polypeptide comprises an amino acid sequence 100% identical to SEQ ID NO: 4 [CfTPS3]. The polypeptide of the invention may comprise a biologically active fragment of at least 50 contiguous amino acids of any of i) through ii), said fragment having at least 75% sequence identity to SEQ ID NO: 4 [CfTPS3] in a range of overlap of at least 50 amino acids, wherein the biological activity is diterpene synthase activity, preferably class I diterpene synthase activity.

In another embodiment the polypeptide of the invention comprises:
i) an amino acid sequence identical to SEQ ID NO: 3 [CfTPS4],
ii) a biologically active sequence variant of said polypeptide, wherein the sequence variant is at least 75% identical to, such as at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 3 [CfTPS4],
iii) a biologically active fragment of at least 50 contiguous amino acids of any of i) through ii), said fragment having at least 75% sequence identity to SEQ ID NO: 3 [CfTPS4] in a range of overlap of at least 50 amino acids, wherein the biological activity is diterpene synthase activity, preferably class I diterpene synthase activity.

In a preferred embodiment, the polypeptide comprises a biologically active sequence variant having an amino acid sequence at least 95% identical to SEQ ID NO: 3 [CfTPS4]. In another preferred embodiment, the polypeptide comprises an amino acid sequence 100% identical to SEQ ID NO: 3 [CfTPS4]. The polypeptide of the invention may comprise a biologically active fragment of at least 50 contiguous amino acids of any of i) through ii), said fragment having at least 75% sequence identity to SEQ ID NO: 3 [CfTPS4] in a range of overlap of at least 50 amino acids, wherein the biological activity is diterpene synthase activity, preferably class I diterpene synthase activity.

In yet another embodiment, embodiment the polypeptide of the invention comprises:
i) an amino acid sequence identical to SEQ ID NO: 5 [EpTPS8],
ii) a biologically active sequence variant of said polypeptide, wherein the sequence variant is at least 75% identical to, such as at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 5 [EpTPS8],
iii) a biologically active fragment of at least 50 contiguous amino acids of any of i) through ii), said fragment having at least 75% sequence identity to SEQ ID NO: 5 [EpTPS8] in a range of overlap of at least 50 amino acids, wherein the biological activity is diterpene synthase activity, preferably class I diterpene synthase activity.

In some embodiments, the polypeptide of the invention comprises an operative class I DDxxD (SEQ ID NO: 12) domain. Preferably, the polypeptides of the invention (in particular the Class I diTPS) or variants or fragments thereof as defined above comprise a DDxxD domain. This domain is found at positions 531-535 of SEQ ID NO: 3 [CfTPS4], 299-303 of SEQ ID NO: 4 [CfTPS3], 540-544 of SEQ ID NO: 5 [EpTPS8]. Other important functional domains of the class I diTPS polypeptides of the invention are Mg²⁺-binding sites, active site lid residues and substrate binding pockets. The relevant residues for each of SEQ ID NO: 4 [CfTPS3], SEQ ID NO: 3 [CfTPS4] and SEQ ID NO: 5 [EpTPS8] are listed in Table 1. Some embodiments of the invention concern polypeptides, such as Class I diTPS in which these residues or domains are not modified.

In some embodiments, the polypeptides of the invention comprise a plastidial target domain. The plastidial target domain is comprised in the domain ranging from positions 1 to 73 of SEQ ID NO: 3 [CfTPS4], 1 to 73 of SEQ ID NO: 4 [CfTPS3], 1 to 73 of SEQ ID NO: 5 [EpTPS8], respectively. Thus preferred polypeptides (e.g. Class I diTPS) comprise the plastidial target domain.

TABLE 1
Important domains and residues for class I diTPS
of the invention.

| | Plastidial | Mg²⁺- | Active | Substrate |
|---|---|---|---|---|
| | targeting | binding | site lid | binding |
| | signal | sites | residues | pockets |
| CfTPS3 | 1-73 | N443, | EKERKENTGNSV | R262 L271 |
| (SEQ ID | | E451 | (449-460); | H292 V294 |
| NO: 4) | | | GDEF | L295 T297 |
| | | | (526-529) | D300 D304 |
| | | | | R440 L441 |
| | | | | N443 D444 |
| | | | | T446 E451 |
| | | | | Y523 G526 |
| | | | | F529 |
| CfTPS4 | 1-73 | N475, | EKERKENTGNSV | R294 L303 |
| (SEQ ID | | E483 | (481-492); | H324 V326 |
| NO: 3) | | | GDEF | L327 T329 |
| | | | (559-562) | D332 D336 |
| | | | | R472 L473 |
| | | | | N475 D476 |
| | | | | T478 E483 |
| | | | | Y556 G559 |
| | | | | F562 |
| EpTPS8 | 1-73 | N684, | KRESAQGKLNGV | R503 T512 |
| (SEQ ID | | E692 | (690-701); | N533 V535 |
| NO: 5) | | | DDGF | F536 T538 |
| | | | (536-538) | D541 D545 |
| | | | | R681 L682 |
| | | | | N684 D685 |
| | | | | T687 E692 |
| | | | | Y764 D767 |
| | | | | F770 |

The residues are indicated as Xn, where X is the one letter amino acid code and n the position of the residue in the relevant sequence, except for the plastidial targeting signal, where the two numbers indicate the positions of the first and last residue of the predicted domain.

Mg²⁺-binding sites for CfTPS3 (SEQ ID NO: 4) are found at positions 443 (N) and 451 (E).

A biologically active sequence variant of a class I diTPS is preferably a polypeptide sharing the above mentioned sequence identity with CfTPS3, CfTPS4 or EpTPS8 and which preferably comprises above-mentioned domains and which is capable of catalysing cleavage of the diphosphate group of LPP and additional cyclization or rearrangement reactions on the resulting carbocation, yielding (13R)-manoyl oxide.

Class II Diterpene Synthases

In one aspect the invention provides a polypeptide having diPTS activity. Said polypeptide is useful in the methods of manufacturing (13R)-manoyl oxide according to the invention,

In some embodiments, the polypeptide has a class II diTPS activity. Class II diTPS are capable of catalysing protonation-initiated cationic cycloisomerization reactions. The reaction is terminated either by deprotonation or by water capture of the diphosphate carbocation. A class II diTPS relevant for the invention is TPS2 from Coleus forskohlii (CfTPS2), which can catalyse cycloisomerisation of GGPP to LPP. Deprotonation or water capture terminate the class II diTPS reaction.

In one embodiment the polypeptide of the invention comprises:
i) an amino acid sequence identical to SEQ ID NO: 1 [CfTPS2],
ii) a biologically active sequence variant of said polypeptide, wherein the sequence variant is at least 75% identical to, such as at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 1 [CfTPS2],
iii) a biologically active fragment of at least 50 contiguous amino acids of any of i) through ii), said fragment having at least 75% sequence identity to SEQ ID NO: 1 [CfTPS2] in a range of overlap of at least 50 amino acids, wherein the biological activity is diterpene synthase activity, preferably class I diterpene synthase activity.

In a preferred embodiment, the polypeptide comprises a biologically active sequence variant having an amino acid sequence at least 95% identical to SEQ ID NO: 1 [CfTPS2]. In another preferred embodiment, the polypeptide comprises an amino acid sequence 100% identical to SEQ ID NO: 1 [CfTPS2]. The polypeptide of the invention may comprise a biologically active fragment of at least 50 contiguous amino acids of any of i) through ii), said fragment having at least 75% sequence identity to SEQ ID NO: 1 [CfTPS2] in a range of overlap of at least 50 amino acids, wherein the biological activity is diterpene synthase activity, preferably class I diterpene synthase activity.

In some embodiments, the polypeptide of the invention comprises an operative class II DxDD domain. Preferably, the polypeptides of the invention (e.g. the Class II diTPS) or variants or fragments thereof as defined above comprise a DxDD domain. This domain is found at positions 358-361 of SEQ ID NO: 1 [CfTPS2].

In some embodiments, the polypeptides of the invention comprise a plastidial target domain. Thus, Class II diTPS may comprise a plastidial target domain. The plastidial target domain is comprised in the domain ranging from positions 1 to 73 of SEQ ID NO: 1 [CfTPS2].

A biologically active sequence variant of a class II diTPS is preferably a polypeptide sharing the above mentioned sequence identity with CfTPS2 or SsLPPS and which preferably comprises above-mentioned domains and which is capable of catalysing cycloisomerisation of GGPP to LPP.

Polynucleotide

The invention further relates to a polynucleotide encoding a polypeptide according to the invention.

In some embodiments, the polynucleotide has a sequence with at least 85% identity to a sequence selected from the group consisting of SEQ ID NO: 6 [CfTPS2], SEQ ID NO: 9 [CfTPS3], SEQ ID NO: 8 [CfTPS4] and SEQ ID NO: 10 [EpTPS8].

In some embodiments, the polynucleotide has a sequence with at least 85% identity, such as at least 90% identity, such as at least 95% identity, such as at least 96% identity, such as at least 97% identity, such as at least 98% identity, such as 99% identity, such as 100% identity to SEQ ID NO: 6 [CfTPS2]. The polynucleotide may in particular encode a polypeptide of SEQ ID NO:1 [CfTPS2] or a biologically active sequence variant thereof sharing at least 75%, such as at least 80%, such as at least 85%, such as at least 90%, such as at least 95%, such as at least 98% sequence identity with SEQ ID NO:1.

In other embodiments, the polynucleotide has a sequence with at least 85% identity, such as at least 90% identity, such as at least 95% identity, such as at least 96% identity, such as at least 97% identity, such as at least 98% identity, such as 99% identity, such as 100% identity to SEQ ID NO: 9 [CfTPS3]. The polynucleotide may in particular encode a polypeptide of SEQ ID NO:4 [CfTPS3] or a biologically active sequence variant thereof sharing at least 75%, such as at least 80%, such as at least 85%, such as at least 90%, such as at least 95%, such as at least 98% sequence identity with SEQ ID NO:4.

In other embodiments, the polynucleotide has a sequence with at least 85% identity, such as at least 90% identity, such as at least 95% identity, such as at least 96% identity, such as at least 97% identity, such as at least 98% identity, such as 99% identity, such as 100% identity to SEQ ID NO: 8 [CfTPS4]. The polynucleotide may in particular encode a polypeptide of SEQ ID NO:3 [CfTPS4] or a biologically active sequence variant thereof sharing at least 75%, such as at least 80%, such as at least 85%, such as at least 90%, such as at least 95%, such as at least 98% sequence identity with SEQ ID NO:3.

In other embodiments, the polynucleotide has a sequence with at least 85% identity, such as at least 90% identity, such as at least 95% identity, such as at least 96% identity, such as at least 97% identity, such as at least 98% identity, such as 99% identity, such as 100% identity to SEQ ID NO: 10 [EpTPS8]. The polynucleotide may in particular encode a polypeptide of SEQ ID NO:5 [EpTPS8] or a biologically active sequence variant thereof sharing at least 75%, such as at least 80%, such as at least 85%, such as at least 90%, such as at least 95%, such as at least 98% sequence identity with SEQ ID NO:5.

Thus the polynucleotides of the invention encode polypeptides having a diTPS activity as described above. Preferably, the polynucleotide comprises a sequence coding for an operative class I domain DDxxD. This is in particular the case, when the polynucleotide encode a class I diTPS. The DDxxD domain of the CfTPS3 diTPS is DDFFD, where D is an aspartic acid and F is a phenylalanine, and is found at positions 330 to 334 (SEQ ID NO: 4). The DDxxD domain of the CfTPS4 diTPS is DDFFD, where D is an aspartic acid and F is a phenylalanine, and is found at positions 331 to 335 (SEQ ID NO: 3). The DDxD domain of the EpTPS8 diTPS is DDFFD, where D is an aspartic acid and F is a phenylalanine, and is found at positions 540 to 544 (SEQ ID NO: 5).

In another preferred embodiment, the polynucleotide comprises a sequence coding for an operative class II domain DxDD. The DxDD domain of the CfTPS2 diTPS is DIDD, where D is an aspartic acid and I is an isoleucine residue, and is found at positions 399 to 402 (SEQ ID NO: 1).

Some polynucleotides of the invention may comprise a sequence coding for a plastidial targeting signal. The plastidial target domain is comprised in the corresponding polypeptides in the domains ranging from positions 1 to 50 of SEQ ID NO: 1 [CfTPS2], 1 to 33 of SEQ ID NO: 3 [CfTPS4], 1 to 3 of SEQ ID NO: 4 [CfTPS3], 1 to 5 of SEQ ID NO: 5 [EpTPS8], respectively.

The polynucleotide may have a sequence that is codon-optimised. Codon optimisation methods are known in the art and allow optimised expression in a heterologous host organism or cell. The host cell may be selected from the group comprising bacterial cell, yeast cells, fungal cells, plant cells, mammalian cells and insect cells. The host cell may be selected from the group comprising Escherichia coli, Saccharomyces cerevisiae, Schizosaccharomyces pombe, Nicotiana benthamiana and Physcomitrella patens. Thus in one embodiment, the host cell is Escherichia coli. In another embodiment, the host cell is Saccharomyces cerevisiae or Schizosaccharomyces pombe. In another embodiment, the host cell is Nicotiana benthamiana. In another embodiment, the host cell is Physcomitrella patens.

Vectors

The invention further relates to a vector comprising at least one polynucleotide as defined above. Thus the invention relates to a vector suitable for expression of at least one polypeptide having a diTPS activity.

In some embodiments, the vector comprises a polynucleotide having a sequence with at least 85% identity to a sequence selected from the group consisting of SEQ ID NO: 6 [CfTPS2], SEQ ID NO: 9 [CfTPS3], SEQ ID NO: 8 [CfTPS4] and SEQ ID NO: 10 [EpTPS8]. In some embodiments, the vector comprises a polynucleotide having a sequence with at least 85% identity, such as at least 90% identity, such as at least 95% identity, such as at least 96% identity, such as at least 97% identity, such as at least 98% identity, such as 99% identity, such as 100% identity to SEQ ID NO: 6 [CfTPS2]. In other embodiments, the vector comprises a polynucleotide having a sequence with at least 85% identity, such as at least 90% identity, such as at least 95% identity, such as at least 96% identity, such as at least 97% identity, such as at least 98% identity, such as 99% identity, such as 100% identity to SEQ ID NO: 9 [CfTPS3]. In other embodiments, the vector comprises a polynucleotide having a sequence with at least 85% identity, such as at least 90% identity, such as at least 95% identity, such as at least 96% identity, such as at least 97% identity, such as at least 98% identity, such as 99% identity, such as 100% identity to SEQ ID NO: 8 [CfTPS4]. In other embodiments, the polynucleotide has a sequence with at least 85% identity, such as at least 90% identity, such as at least 95% identity, such as at least 96% identity, such as at least 97% identity, such as at least 98% identity, such as 99% identity, such as 100% identity to SEQ ID NO: 10 [EpTPS8].

In some embodiments, the vector comprises a first polynucleotide having a sequence with at least 85% identity to a sequence selected from the group consisting of SEQ ID NO: 6 [CfTPS2], SEQ ID NO: 9 [CfTPS3], SEQ ID NO: 8 [CfTPS4] and SEQ ID NO: 10 [EpTPS8] and a second polynucleotide having a sequence with at least 85% identity to a sequence selected from the group consisting of SEQ ID NO: 6 [CfTPS2], SEQ ID NO: 9 [CfTPS3], SEQ ID NO: 8 [CfTPS4] and SEQ ID NO: 10 [EpTPS8], where the second polynucleotide is different from the first polynucleotide. In preferred embodiments, the vector comprises a first polynucleotide coding for a polypeptide with a class I diTPS activity and a second polynucleotide coding for a polypeptide with a class II diTPS activity. In a preferred embodiment, the first polynucleotide has a sequence with at least 85% identity to SEQ ID NO: 6 [CfTPS2], and the second polynucleotide has a sequence with at least 85% identity to SEQ ID NO: 9 [CfTPS3], SEQ ID NO: 8 [CfTPS4] and SEQ ID NO: 10 [EpTPS8].

Vectors of the invention comprise plasmids, cosmids, viral vectors, artificial chromosomes and bacterial vectors. The vector may be suitable for transient expression of the at least one polynucleotide. Such vectors are known in the art. For example, expression may be induced by addition of a compound to the growth medium. The vector may be a bacterial vector, such as Agrobacterium tumefaciens. In a preferred embodiment, the vector also encodes a viral suppressor of gene silencing, such as the p19 protein of tomato bushy stunt virus.

Host Cell

The invention further relates to a host cell comprising a polynucleotide as defined above and/or a vector as defined above. In some embodiments, the host cell is capable of producing (13R)-manoyl oxide.

The host cell of the invention is capable of expressing:
(i) a first polypeptide having a sequence at least 70% identical to SEQ ID NO: 1 [CfTPS2]; and
(ii) a second polypeptide having a sequence at least 70% identical to SEQ ID NO: 4 [CfTPS3], SEQ ID NO: 3 [CfTPS4], or SEQ ID NO: 5 [EpTPS8].

In some embodiments, the host cell is capable of expressing a first polypeptide having a class I diTPS activity and a second polypeptide having a class II diTPS activity.

Thus in some embodiments the host cell is capable of expressing a first polypeptide having a sequence at least 70% identical to SEQ ID NO: 1 [CfTPS2] and a second polypeptide having a sequence at least 70% identical to SEQ ID NO: 4 [CfTPS3]. In particular, the host cell is capable of expressing a first polypeptide, which is CfTPS2 of SEQ ID NO:1 or a biologically active variant thereof sharing at least 75%, such as at least 85%, such as at least 95% sequence identity with SEQ ID NO: 1 [CfTPS2] and a second polypeptide, which is CfTPS3 of SEQ ID NO:4 or a biologically active variant thereof sharing at least 75%, such as at least 85%, such as at least 95% sequence identity with SEQ ID NO:4 [CfTPS3].

In other embodiments, the host cell is capable of expressing a first polypeptide having a sequence at least 70% identical to SEQ ID NO: 1 [CfTPS2] and a second polypeptide having a sequence at least 70% identical to SEQ ID NO: 3 [CfTPS4]. In particular, the host cell is capable of expressing a first polypeptide, which is CfTPS2 of SEQ ID NO:1 or a biologically active variant thereof sharing at least 75%, such as at least 85%, such as at least 95% sequence identity with SEQ ID NO: 1 [CfTPS2] and a second polypeptide, which is CfTPS4 of SEQ ID NO:3 or a biologically active variant thereof sharing at least 75%, such as at least 85%, such as at least 95% sequence identity with SEQ ID NO:3 [CfTPS4].

In other embodiments, the host cell is capable of expressing a first polypeptide having a sequence at least 70% identical to SEQ ID NO: 1 [CfTPS2] and a second polypeptide having a sequence at least 70% identical to SEQ ID NO: 5 [EpTPS8]. In particular, the host cell is capable of expressing a first polypeptide, which is CfTPS2 of SEQ ID NO:1 or a biologically active variant thereof sharing at least 75%, such as at least 85%, such as at least 95% sequence identity with SEQ ID NO: 1 [CfTPS2] and a second polypeptide, which is EpTPS8 of SEQ ID NO:5 or a biologically active variant thereof sharing at least 75%, such as at least 85%, such as at least 95% sequence identity with SEQ ID NO:5 [EpTPS8].

The host cell may be selected from the group comprising bacterial cell, yeast cells, fungal cells, plant cells, mammalian cells and insect cells. The host cell may be selected from the group comprising Escherichia coli, Saccharomyces cerevisiae, Schizosaccharomyces pombe, Nicotiana benthamiana and Physcomitrella patens. Thus in one embodiment, the host cell is Escherichia coli. In another embodiment, the host cell is Saccharomyces cerevisiae or Schizosaccharomyces pombe. In another embodiment, the host cell is Nicotiana benthamiana. In another embodiment, the host cell is Physcomitrella patens.

Methods for expressing the first and second polypeptides in the host cell are known in the art. One or both of the first and second polypeptides may be heterologously expressed from polynucleotide sequences cloned into the genome of the host cell or they may be comprised within a vector as described above. For example, a first polynucleotide coding for the first polypeptide is cloned into the genome, and a second polynucleotide coding for the second polypeptide is comprised within a vector transformed or transfected into the host cell. Alternatively, the first polynucleotide is comprised within a first vector and the second polynucleotide is comprised within a second vector. The first and second vector may be one vector. Vectors suitable for expression of the first and second polypeptides are known in the art.

Expression of the first and second polypeptides in the host cell may occur in a transient manner. When the polynucleotide encoding one of the polypeptides is cloned into the genome, an inducible promoter may be cloned as well to control expression of the polypeptides. Such inducible promoters are known in the art. Alternatively, genes coding for suppressors of gene silencing may also be cloned in the genome or into a vector transfected within the host cell.

The host cell may be selected from the group comprising bacterial cell, yeast cells, fungal cells, plant cells, mammalian cells and insect cells. The host cell may be selected from the group comprising Escherichia coli, Saccharomyces cerevisiae, Schizosaccharomyces pombe, Nicotiana benthamiana and Physcomitrella patens. Thus in one embodiment, the host cell is Escherichia coli. In another embodiment, the host cell is Saccharomyces cerevisiae or Schizosaccharomyces pombe. In another embodiment, the host cell is Nicotiana benthamiana. In another embodiment, the host cell is Physcomitrella patens.

In some embodiments, the host cell is transfected with a vector. The vector may be selected from the group comprising plasmids, cosmids, viral vectors, artificial chromosomes and bacterial vectors. The vector may be constructed so that transient expression of the first and/or second polynucleotide from the vector is transient. Transient expression from the vector may occur via induction of an inducible promoter as is known in the art. In a preferred embodiment, the host cell is a Nicotiana benthamiana cell, such as a leaf cell. In another preferred embodiment, the vector is the bacterial vector Agrobacterium tumefaciens. In a preferred embodiment, the vector also encodes a viral suppressor of gene silencing. In a most preferred embodiment, the vector encodes the p19 protein from tomato bushy stunt virus.

The host cell may naturally be capable of producing GGPP. In other embodiments, the cell may be engineered so that it is capable of producing GGPP. For example, the plastidial methylerythritol 4-phosphate (MEP) pathway may be cloned into the host cell. Thus, the host cell may comprise one or more heterologous nucleic acids encoding one or more enzymes of the MEP pathway.

The host cell may be further engineered in order to redirect metabolic fluxes and optimise for the production of a specific compound.

In some embodiments, the host cell is capable of producing diterpenoids. The host cell may be capable of producing (13R)-manoyl oxide in a substantially pure enantiomeric form.

### EXAMPLES

### Example 1

### Identification of Coleus forskohlii diTPS Genes

Forskolin is a representative of an unusual series of tricyclic (8,13)-epoxy-labdanes, characteristic for this plant. Given its importance as a pharmaceutical, we set out to discover genes involved in the biosynthesis of forskolin.

Materials and Methods

Plant Growth and Microscopy

Coleus forskohlii (Lamiaceae) plants were grown in the greenhouse at the University of Copenhagen, Denmark, under ambient photoperiod and 240 day/17° C. night temperatures. Transverse sections of roots (diameter of approximately 1 to 5 mm) were prepared for histochemical analysis. Sections were observed unstained with a Leica DM 5000B or a Nikon Eclipse 80i light and fluorescence microscope.

Additionally, root samples were fixed in a solution containing 2.5% glutaraldehyde, 2% paraformaldehyde, and 0.1 M sodium cacodylate buffer, pH 7.2 for 24 h, thereafter surface sections and cross sections from the root cork were incubated in 0.1 μg/mL Nile Red for identification of lipid components. Images from intact cells were recorded in a Leica SP5X confocal laser scanning microscope (Leica Microsystems, Mannheim, Germany).

Diterpene Profiling and Forskolin Quantification in C. forskohlii Tissues

Tissue was extracted. Cold methanol acidified with formic acid (0.125%) was added to ground and frozen tissue samples in a ratio of 3:1 (solvent:tissue). Samples were sonicated in an ultrasonic bath at 23° C. for 15 min at 40 kHz (Branson, 3510), filtered using 96 well filter plates and analyzed by HPLC (High-Performance Liquid Chromatography) equipped with an evaporative light scattering detector (ELSD). All tissue types were extracted in triplicate. Forskolin was quantified by comparison to a standard series of forskolin (Sigma) which was prepared as per the tissue samples.

For the diterpene profiling of the root cork HPLC-MS, extracts were prepared as for forskolin quantification. Separation was carried out on an Agilent 1100 Series LC unit (Agilent Technology) with column and gradient as described above. The LC unit was coupled to either a Bruker microTOF mass spectrometer (Bruker Daltronics) for high resolution mass measurements or a Bruker HCT-Ultra ion trap mass spectrometer (Bruker Daltronics) for MSn experiments.

Isolation of C. forskohlii Root Cork Oil Bodies

For isolation of oil bodies from root cork tissue, approximately 15 g of tissue was gently ground in 100 mL extraction buffer (EB; 20 mM Tricine, 250 mM sucrose, 0.2 mM PMSF, pH 8.5); the homogenate was filtered through Miracloth (Calbiochem) and centrifuged at 3500 rpm for 10 min for separation of cellular debris. The supernatant was collected and transferred in centrifugation tubes. Buffer B (20% sucrose, 20 mM HEPES, 100 mM KCl, 2 mM MgCl₂, pH 10.5) was overlaid (5 mL of B for 25 mL of supernatant) and samples were centrifuged for 40 min at 5000 g. The resulting floating oil bodies were collected carefully from the surface layer.

Identification and Cloning of Full Length diTPS Genes

Mining of the C. forskohlii databases was performed as previously described (Zerbe et al., 2013), using tBLASTn software and known angiosperm diTPSs as query (CPS and EKS) and guided full-length cloning of a number of putative class I and class II diTPS genes. Total RNA from C. forskohlii roots, extracted as previously described (Hamberger et al., 2011), was used for cDNA synthesis. Cloning of the putative diTPS genes was achieved after PCR amplification using gene specific primers (SEQ ID NO: 13 to 42) that were designed based on the in silico sequences of the identified CfTPS genes.

RNA Extraction and Quantitative Real-Time PCR

Total RNA from C. forskohlii root cork was extracted according to Hamberger et al., 2011, and further purified using the Spectrum Plant Total RNA Kit (Sigma) while total RNA from leaves, flowers, stems and root cortex and stele was extracted using the Spectrum Plant Total RNA Kit (Sigma). RNA extraction was followed by on-column DNase I digestion. First-strand cDNAs were synthesized from 0.5 μg of total RNA. The resulting cDNA was diluted 10-fold for the qRT-PCR reactions. Quantitative real-time PCR reactions were performed with gene specific primers (SEQ ID NO: 43 to 62) and Maxima SYBR Green/Fluorescein qPCR Master Mix (Fermentas) on a Rotor-Gene Q cycler (Qiagen). TIF4a and EF1a were used as reference genes as they showed the lowest variation across different tissues. The results were normalized with TIF4a. Relative transcript abundance was calculated as the mean of three biological replications (three different plants), while the reactions were performed in three technical replicates. Amplification efficiency was calculated with the “Real Time PCR Miner” (http://www.miner.ewindup.info/Version2). Efficiency-corrected ΔC_{T} values were used to quantify relative differences in target gene transcript accumulation.

Functional Characterization of CfTPS—In Vitro Assays

For the expression of CfTPS1, CfTPS2, CfTPS3, CfTPS4 and CfTPS14 in E. coli, pseudomature variants lacking predicted plastidial target sequences were cloned into the into pET28b+ vector. The software ChloroP was used for prediction of the plastidial target sequence (http://www.cbs.dtu.dk/services/ChloroP/). As the expression levels of the recombinant CfTPS3 were very poor, a codon optimized version was synthesized by GenScript USA Inc. and subsequently cloned into the same vector (SEQ ID NO: 63). pET28b+ constructs were transformed into E. coli BL-21 DE3-C41 cells and grown in selection medium until the OD reached 0.3-0.4. Expression was induced at OD₆₀₀ ˜0.6 with 0.2 mM IPTG. Expression was done overnight and cells were harvested by centrifugation and lysed. The cell lysates were centrifuged and the supernatant was subsequently used for purification of the recombinant proteins. CfTPS proteins were purified on 1 mL His SpinTrap™ columns (GE healthcare). In vitro CfTPS assays were performed by adding 15 μM GGPP and 100 μg purified CfTPS enzymes in 397 μL enzyme assay buffer (50 mM HEPES (pH 7.2), 7.5 mM MgCl₂, 5% (v/v) glycerol, 5 mM DTT). Onto the reaction mix, 500 μL n-hexane (Fluka GC-MS grade) was gently added as an overlay. Assays were incubated for 60 min at 30° C. and −70 rpm and the hexane overlay was subsequently removed for GC-MS analysis.

Functional Characterization of CfTPS—Transient Expression in Nicotiana benthamiana

Full length CfTPS cDNAs were cloned into the agrobacterium binary vector for plant transformation pCAMBIA1300_35Su. Transient expression of CfTPSs with the gene silencing suppressor p19 protein in N. benthamiana leaves and extraction of diterpenes were performed as recently described. Hexane extracts of N. benthamiana expressing the gene silencing suppressor p19 protein alone were used as controls. Compounds of interest were identified by comparison of GC-MS total ion chromatogram (TIC) and extracted ion chromatograms (EIC) of 275 and 272 m/z from samples. The ion 275 m/z is characteristic of several labdane type diterpenes including manoyl oxide whereas 272 m/z is characteristic of several other non-labdane type diterpenes such as abietane like diterpenes. All extractions from N. benthamiana transiently expressing diTPSs were carried out in biological triplicates (different leaves/plants infiltrated with the same agrobacteria mixture).

Metabolite Analysis from In Vitro and in Planta Assays

For the gas chromatography-mass spectrometry (GC-MS) analysis of N. benthamiana leaves expressing the CfTPSs and specific C. forskohlii tissues, 500 μL GC-MS grade hexane was added to 2 leaf discs (Ø=3 cm) in a 1.5 mL glass vial. After extraction, the solvent was transferred into new 1.5 mL glass vials and stored at −20° C. until GC-MS analysis. Compound identification was done by comparison to authentic standards (dehydroabietadiene, abietadiene), reference spectra from literature, databases and comparison of retention time (miltiradiene, manoyl oxide, copalol, labd-13-en-8,15-diol and 13(16)-14-labdien-8-ol) (Wiley Registry of Mass Spectral Data, 8th Edition, July 2006, John Wiley & Sons, ISBN: 978-0-470-04785-9). The differentiation of the C-13 epimers (13R) and (13S)-manoyl oxide was performed as previously described (Demetzos et al., 2002).

Accession Numbers

Nucleotide sequences of characterized enzymes have been submitted to the GenBank™/EBI Data Bank with accession numbers: CfTPS1, KF444506; CfTPS2, KF444507; CfTPS3, KF444508; CfTPS4, KF444509; CfTPS15, KF471011.

### Example 2

### Localization of Forskolin and (13R)-manoyl oxide in Root Cork Oil Bodies

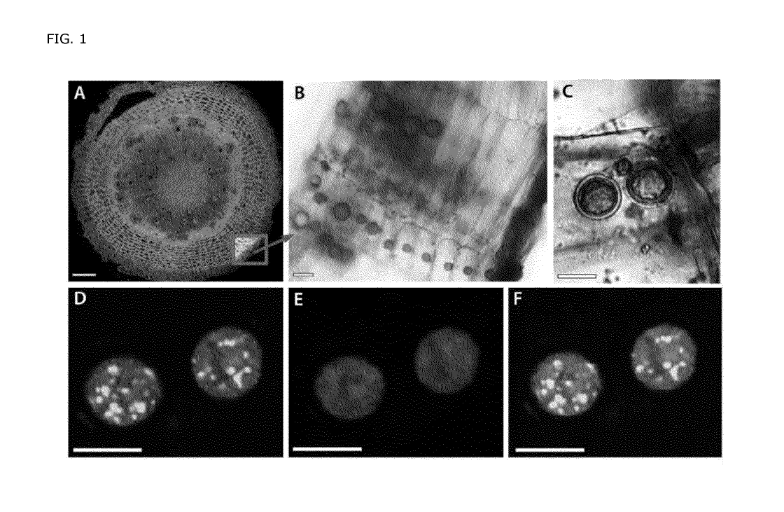

When transverse sections of C. forskohlii root (FIG. 1A) were examined using light microscopy, we found that cells of the root cork contained oil body-like structures (hereafter termed oil bodies) with a typical distribution of one oil body per cell of the root cork (FIG. 1B). Cells containing more than one oil body were occasionally seen in older tissue (FIG. 1C). Confocal laser scanning microscopy of C. forskohlii root cork stained with Nile Red indicated that the observed structures were indeed oil bodies and that the composition of the lipophilic content was heterogeneous, with both polar (FIG. 1E) and neutral (FIG. 1D) lipophilic compounds, which were non-uniformly distributed. Globules of neutral lipids dispersed in predominantly polar lipids were detected by the fluorescence (FIG. 1D-F).

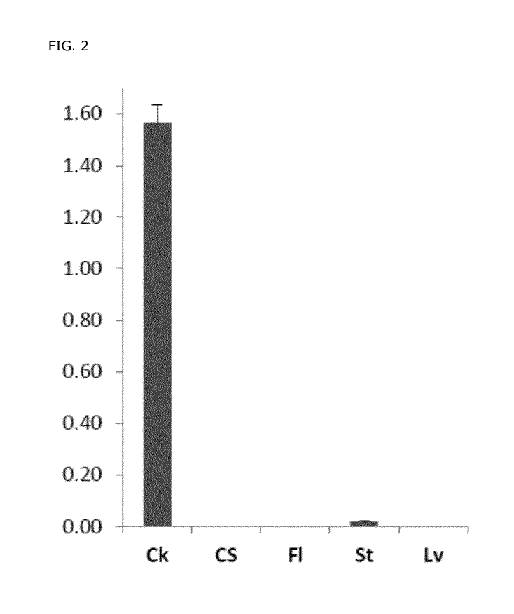

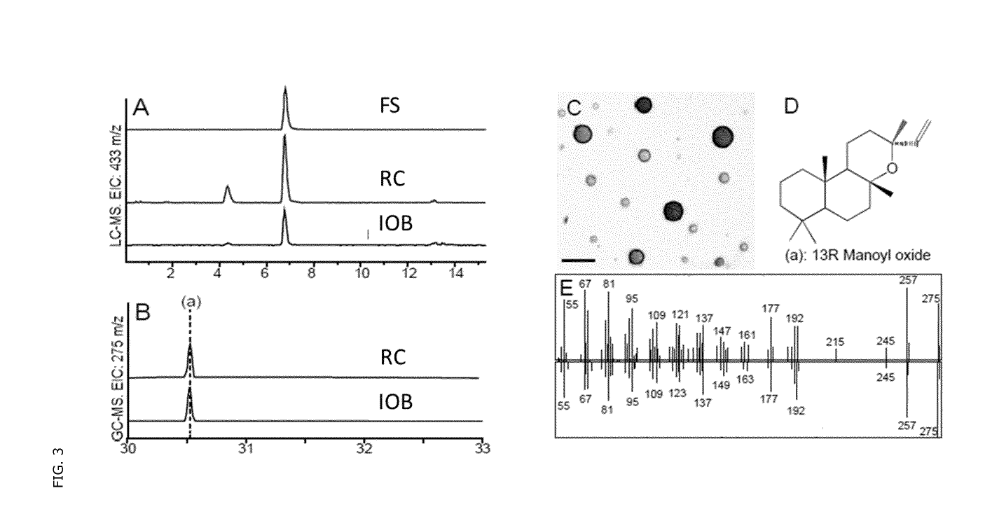

Separate methanol extracts of the root cork and the root stele and cortex were analysed by high-performance liquid chromatography (HPLC) using an evaporative light scattering detector (ELSD) and compared with flowers, leaves and stems. Forskolin was primarily detected in the root cork, and was not found in the root stele and cortex nor in the aerial parts of the plant (FIG. 2). To further examine if forskolin was present specifically in the oil bodies, methanol extracts of isolated oil bodies (oil bodies purified to apparent homogeneity) were subjected to HPLC-ELSD analysis, targeting polar constituents, while non-polar hexane extracts were analyzed by gas chromatography-mass spectrometry (GC-MS) (FIG. 3). In addition to forskolin, which was present in the polar fraction, we detected (13R)-manoyl oxide in the non-polar fraction.

These results demonstrate that (13R)-manoyl oxide and forskolin are present in the oil bodies of C. forskohlii.

### Example 3

### C. forskohlii diTPSs Constitute a Small Gene Family Specific for Lamiaceae

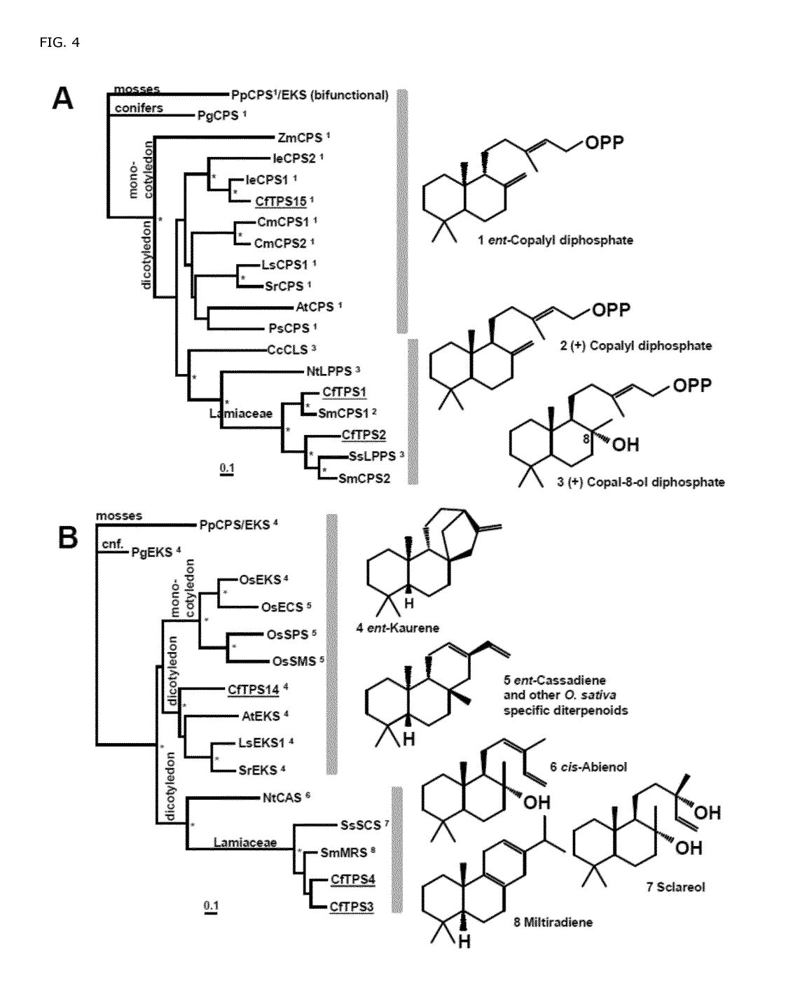

We mined the root transcriptome of C. forskohlii for the identification of CfdiTPS candidates as described (Zerbe et al., 2013). A panel of six diterpene synthases was identified, CfTPS1, CfTPS2, CfTPS3, CfTPS4, CfTPS14 and CfTPS15 which, with exception of CfTPS15, represented full-length cDNAs with predicted N-terminal plastidial transit peptides. CfTPS1, CfTPS2, and CfTPS15 contained the Asp-rich conserved motif DxDD characteristic of class II diTPS, while CfTPS3, CfTPS4 and CfTPS14 carried the DDxxD signature motif of class I diTPS. We performed separate phylogenetic analyses of class II and class I CfTPSs including functionally characterized representatives from the Lamiaceae and other angiosperm species. Included in the phylogenies were representative gymnosperm class II and class I diTPSs [PgCPS and PgEKS] and the bifunctional diTPS from the moss Physcomitrella patens [PpCPS/EKS], as it is considered an ancestral archetype of plant diTPSs (FIG. 4).

Thus we demonstrate that CfTPS1, CfTPS2, and CfTPS15 are phylogenetically related to class II diTPS and that CfTPS3, CfTPS4 and CfTPS14 are phylogenetically related to class I diTPS.

### Example 4

### Transcript Levels of C. forskohlii diTPSs in Various Tissues and In Vitro Functional Characterization

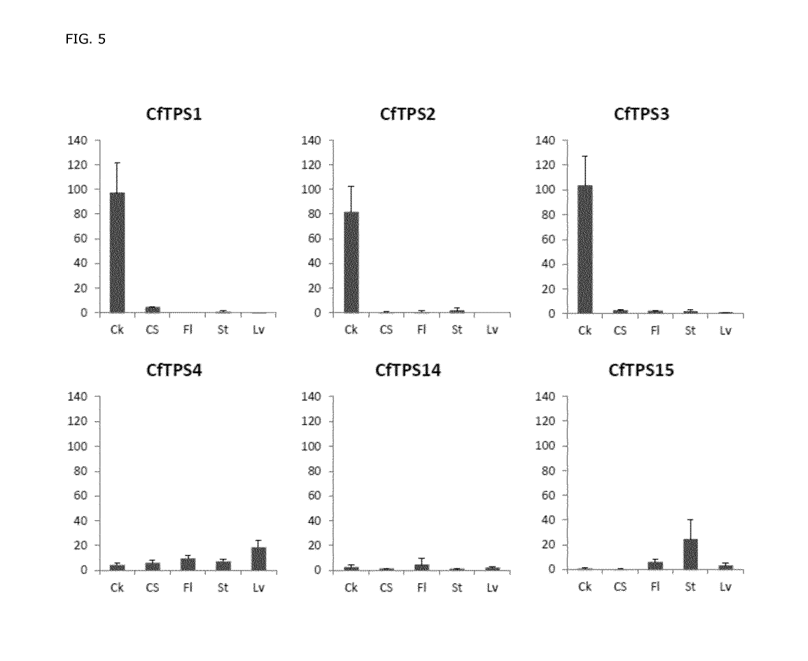

To correlate the transcript levels of CfTPS genes with accumulation of forskolin related labdane-diterpenoids and abietane diterpenoids in C. forskohlii tissues, quantitative (q)RT-PCR analysis was performed using cDNA templates derived from the root cork (Ck), root cortex and stele (root without cork) (CS), leaves (Lv), stems (St) and flowers (FI) total RNA. CfTPS1, CfTPS2 and CfTPS3 shared a similar pattern of transcript profiles across all tissues, showing high transcript accumulation in root cork cells, up to 1000-fold in comparison with all other tissues tested (FIG. 5). These data supported involvement of CfTPS1, CfTPS2 and CfTPS3 in the formation of specialized metabolites in the root cork. In contrast, the transcript levels of CfTPS4, CfTPS14 and CfTPS15 were relatively low across all tissues tested. Despite the close phylogenetic relation of CfTPS3 and CfTPS4 (FIG. 4), they show surprisingly different expression patterns. In contrast to CfTPS3, CfTPS4 transcripts were mostly detected in the aerial parts of the plant, especially in the leaves, while its transcripts accumulate only to very low levels in the root.

For the functional characterization of the CfTPSs described here (except for CfTPS15, for which no full length sequence could be retrieved), cDNAs were heterologously expressed in E. coli with a C-terminal 6×His epitope tag. Purified recombinant proteins were tested individually in single or coupled in vitro assays, supplied with appropriate substrates and the reaction products were analyzed by GC-MS. Products of the in vitro assays with the class II diTSPs, CfTPS1 and CfTPS2, were treated with alkaline phosphatase before GC-MS analysis.

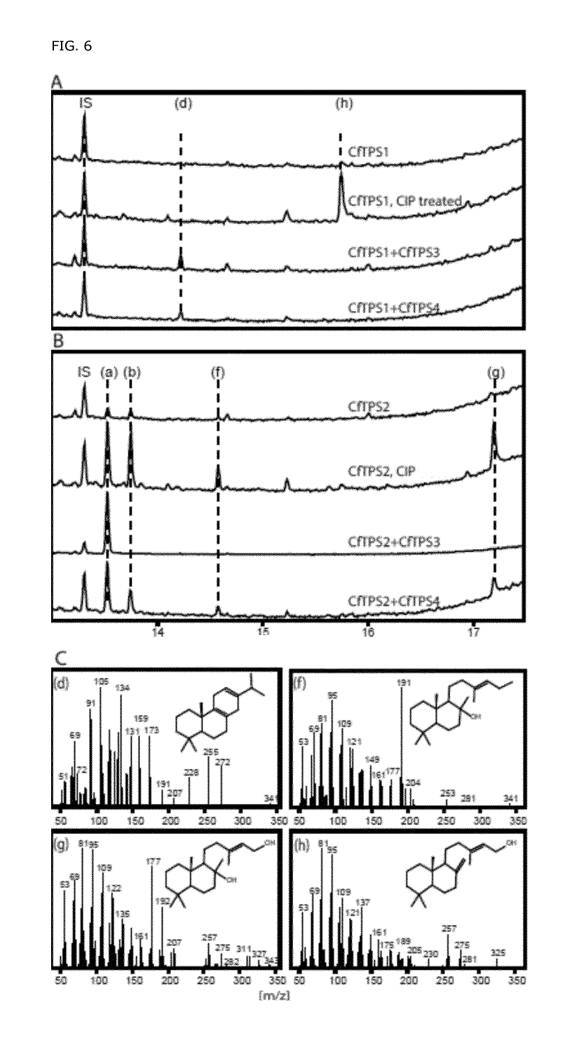

Enzyme assays with CfTPS1 yielded a diterpene with a mass spectrum matching copal-15-ol (h), indicating that the primary product before dephosphorylation is copalyl diphosphate (FIG. 6A). Assays of CfTPS2 resulted in the formation of 13(16)-14-labdien-8-ol (f) and labd-13-en-8,15-diol (g) as major products (FIG. 6B), supporting a function as labda-13-en-8-ol (or copal-8-ol) diphosphate synthase, similar to the functions of previous reported similar enzymes. We also detected the non-stereoselective formation of the (13R) and (13S) epimers of manoyl oxide, which were previously observed in in vitro reactions of similar class II diTPSs and were suggested to be the result of a non-enzymatic reaction (Caniard et al., 2012; Zerbe et al., 2013). These results indicate that CfTPS1 and CfTPS2 represent functionally distinct class II diTPSs, both necessary and sufficient to form the diphosphate intermediates required for the abietane and labdane classes of diterpenoids detected in C. forskohlii.

Assays of CfTPS1 coupled to either CfTPS3 or CfTPS4 resulted in formation of miltiradiene (d) (FIG. 6A), a labdane diterpene formed from a copalyl diphosphate intermediate (Gao et al., 2009) and is consistent with the results of the single enzyme assay of CfTPS1. Coupled assays with CfTPS2 and CfTPS3 showed the formation of the pure (13R) enantiomer of manoyl oxide (a) (FIG. 6B). In coupled assays of CfTPS2 with CfTPS4, both (13R) and (13S)-manoyl oxide epimers were detected, albeit at a ratio different from the dephosphorylation product of CfTPS2 alone (FIG. 6B). The (13R)-manoyl oxide epimer was produced stereospecifically in the combination of class II CfTPS2 and class I CfTPS3 enzymes.

These results demonstrate that the CfTPSs can be expressed heterologously in Escherichia coli. We further show that (13R)-manoyl oxide can be produced stereospecifically in a substantially pure enantiomeric form in an in vitro system.

### Example 5

### In Planta Heterologous Expression and Functional Characterization of C. forskohlii diTPSs

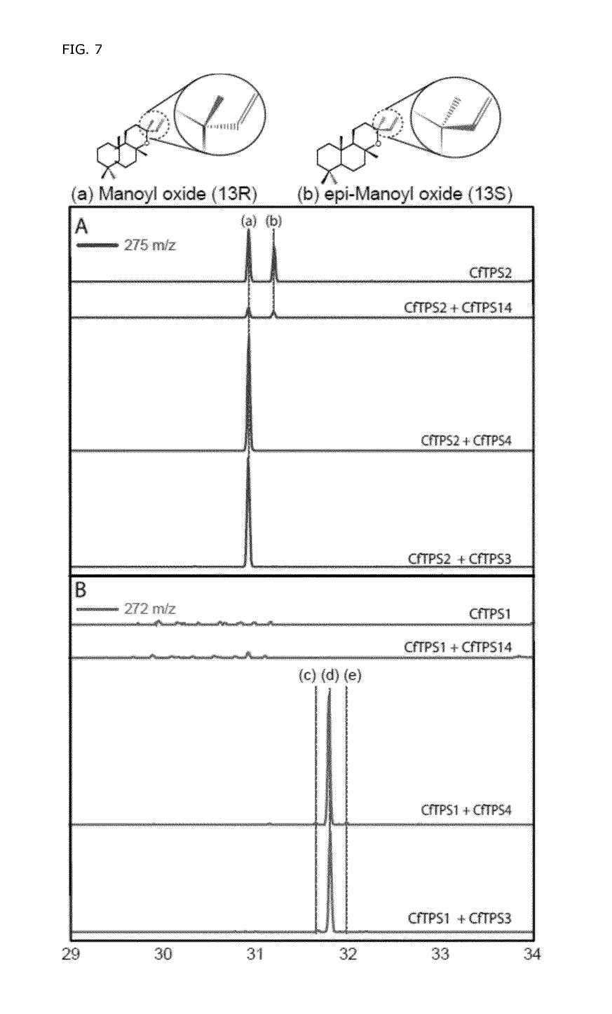

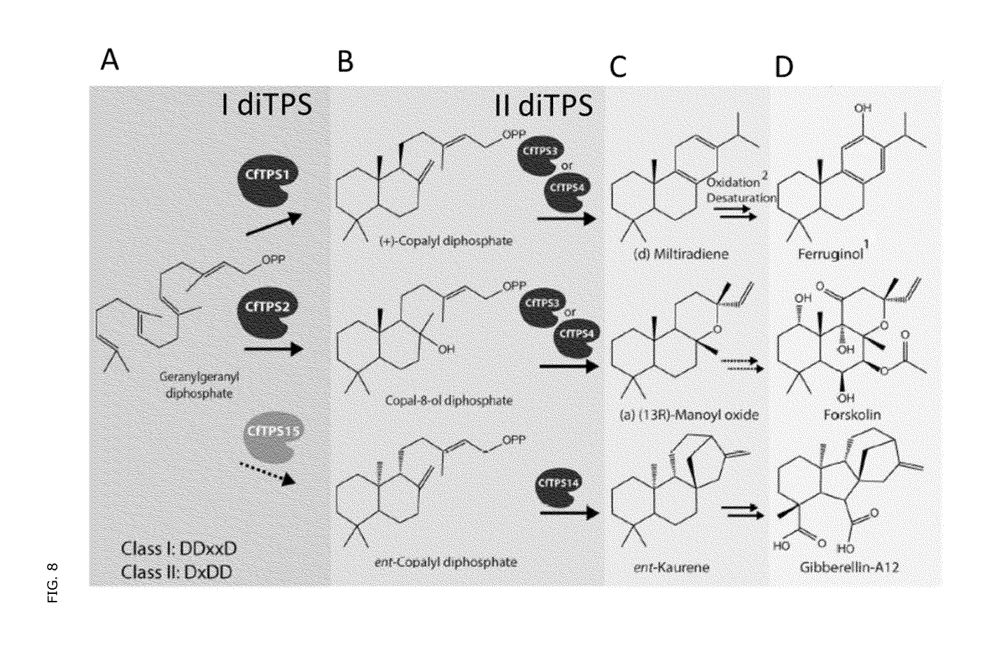

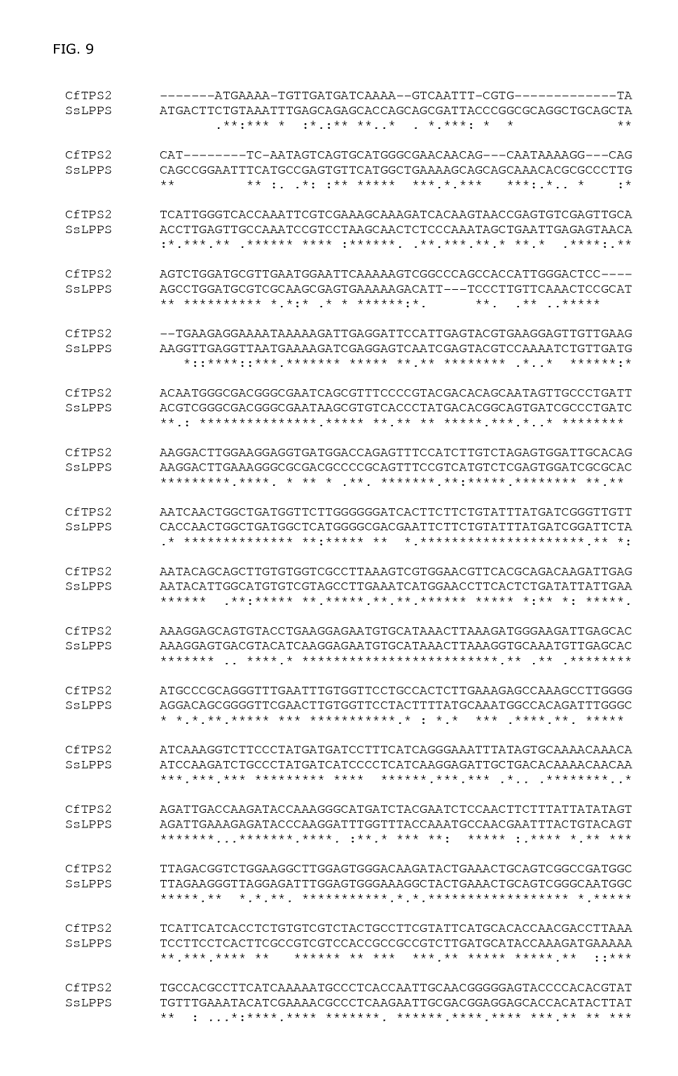

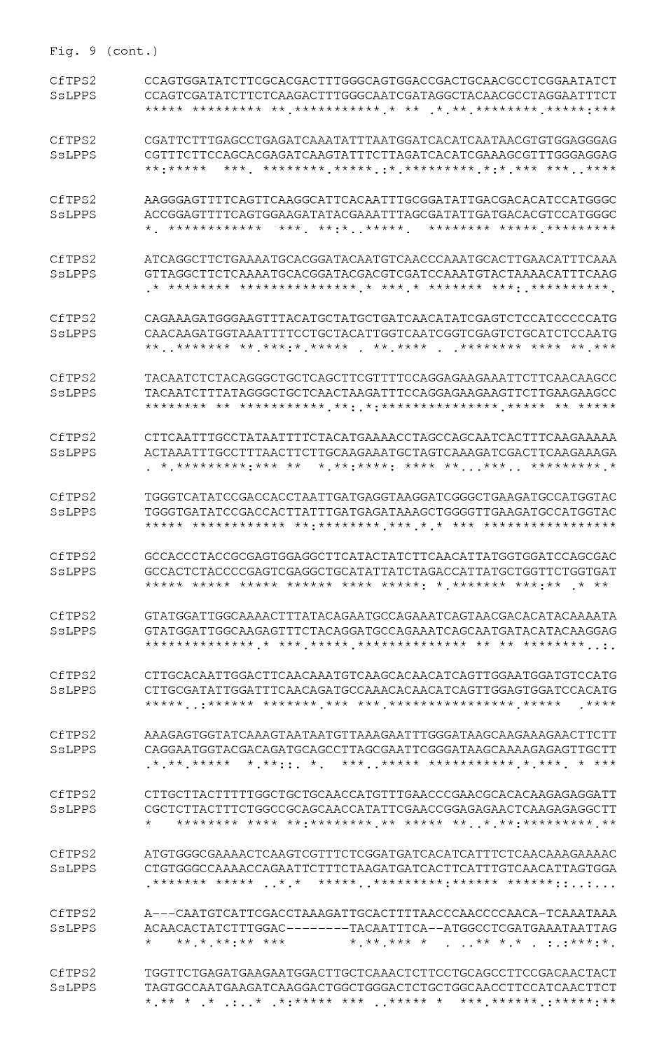

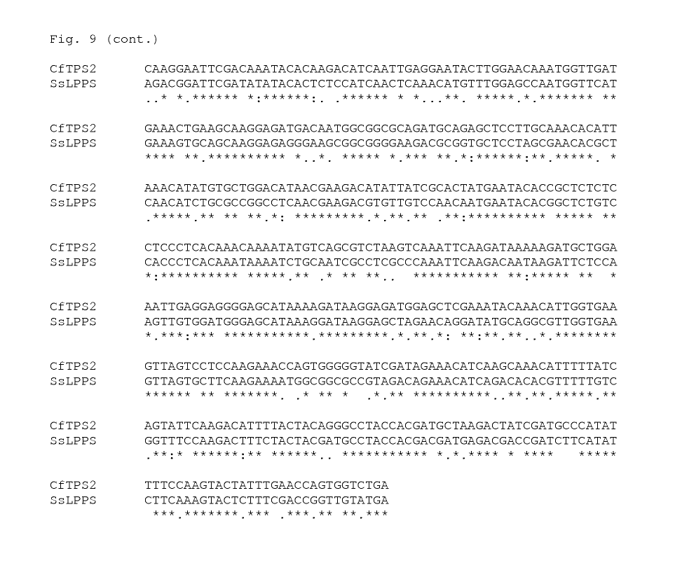

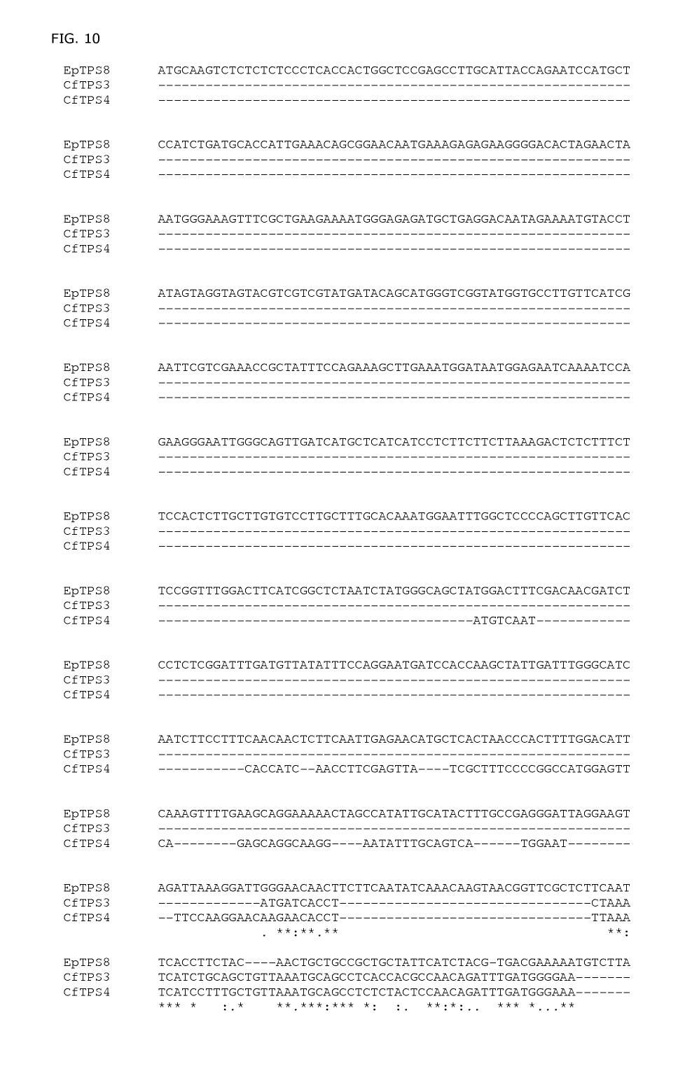

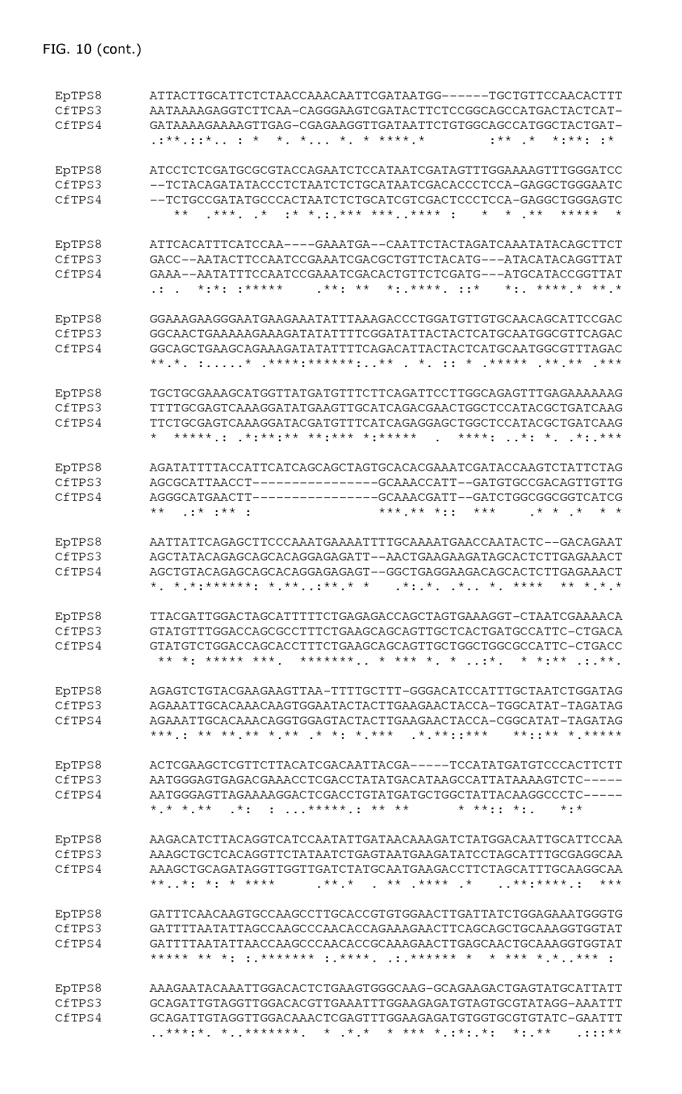

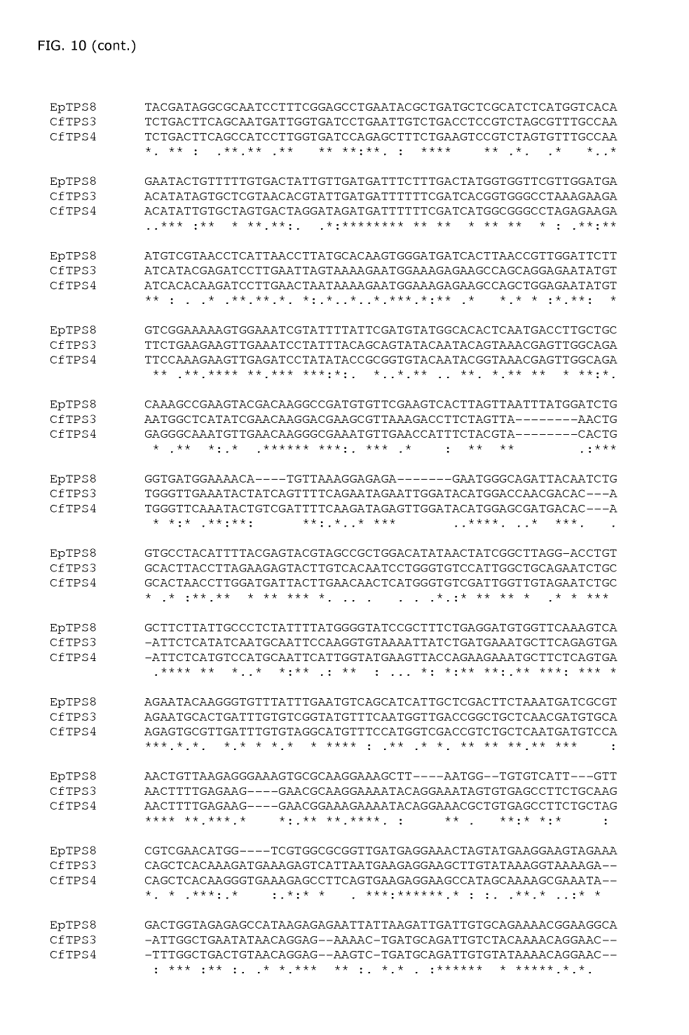

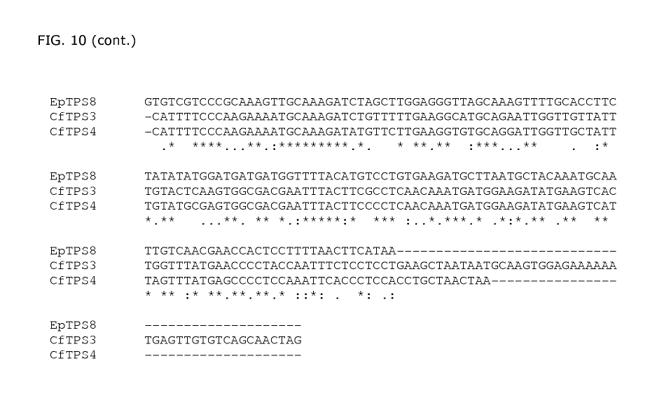

The CfTPSs were expressed in Nicotiana benthamiana leaves after agroinfiltration. GC-MS analyses of extracts from N. benthamiana leaves transiently expressing the individual class I CfTPS3, CfTPS4 and CfTPS14 did not result in detectable accumulation of additional metabolites compared to control plants (data not shown). Extracts from N. benthamiana expressing the class II CfTPS1 alone showed only trace amounts of additional diterpenes compared to the controls, none of which could be accurately identified (FIG. 7B).

Consistent with the in vitro enzyme assays, both (13R) and (13S) epimers of manoyl oxide were identified in the extracts from N. benthamiana expressing the class II CfTPS2 (FIG. 7A). Co-expression of CfTPS2 and CfTPS14 did not change the product profile compared to expression of CfTPS2 alone, suggesting that CfTPS14 does not accept the copal-8-ol diphosphate as substrate (FIG. 7A).

In extracts of plants co-expressing CfTPS1 with CfTPS3 or CfTPS4 miltiradiene (d) was observed as the main product together with minor traces of dehydroabietadiene (c) and abietadiene (e) (FIG. 7B). All three diterpenes were subsequently identified in stem and root tissues of C. forskohlii. While dehydroabietadiene was found in both tissues, abietadiene was mainly detected in the root cork tissue, and miltiradiene in the stem and leaf tissue of C. forskohlii.

In extracts from N. benthamiana co-expressing CfTPS2 with CfTPS3 or CfTPS4, only the (13R) epimer of manoyl oxide (a) was identified (FIG. 7A), consistent with the stereochemical conformation of forskolin and the related series of labdane-type diterpenoids. This result suggests that the class I CfTPS3 and CfTPS4 can accept the copal-8-ol diphosphate synthesized by CfTPS2 and catalyze the stereospecific formation of (13R)-manoyl oxide.

These results demonstrate that the CfTPSs can be agroinfiltrated in a plant organism and that (13R)-manoyl oxide can be produced stereospecifically in a substantially pure enantiomeric form in an in vivo system.

### REFERENCES

Caniard et al., 2012. Discovery and functional characterization of two diterpene synthases for sclareol biosynthesis in Salvia sclarea (L.) and their relevance for perfume manufacture. BMC Plant Biol. 12:119.
Demetzos et al., 2002. A simple and rapid method for the differentiation of C-13 manoyl oxide epimers in biologically important samples using GC-MS analysis supported with NMR spectroscopy and computational chemistry results. Bioorg Med Chem Lett. 12(24):3605-9.
Günnewich et al., 2013. A diterpene synthase from the clary sage Salvia sclarea catalyzes the cyclization of geranylgeranyl diphosphate to (8R)-hydroxy-copalyl diphosphate. Phytochemistry 91:93-9
Hamberger et al., 2011. Evolution of diterpene metabolism: Sitka spruce CYP720B4 catalyzes multiple oxidations in resin acid biosynthesis of conifer defense against insects. Plant Physiol. 157(4):1677-95.
Zerbe et al., 2013. Gene discovery of modular diterpene metabolism in nonmodel systems. Plant Physiol. 162(2):1073-91.

### Items

1. A method of manufacturing (13R)-manoyl oxide, said method comprising the steps of:
(i) providing geranylgeranyl diphosphate (GGPP);
(ii) contacting GGPP of step (i) with a first polypeptide having a sequence at least 70% identical to SEQ ID NO: 1 [CfTPS2] or SEQ ID NO: 2 [SsLPPS], thus obtaining labd-13-en-8,15-diol diphosphate (LPP);
(iii) contacting the LPP of step (ii) with a second polypeptide having a sequence at least 70% identical to SEQ ID NO: 3 [CfTPS4], SEQ ID NO: 4 [CfTPS3] or SEQ ID NO: 5 [EpTPS8];
thus obtaining (13R)-manoyl-oxide.
2. The method according to item 1, wherein the (13R)-manoyl oxide obtained is substantially pure.
3. The method according to any one of the preceding items, wherein the (13R)-manoyl oxide obtained is enantiomerically pure.
4. The method according to any one of the preceding items, wherein the product obtained is essentially free of (13S)-manoyl oxide.
5. The method according to any one of the preceding items, wherein the first polypeptide is CfTPS2 of SEQ ID NO:1 or a biologically active variant thereof sharing at least 75% sequence identity therewith.
6. The method according to any one of the preceding items, wherein the first polypeptide is CfTPS2 of SEQ ID NO:1 or a biologically active variant thereof sharing at least 85% sequence identity therewith.
7. The method according to any one of the preceding items, wherein the second polypeptide is CfTPS3 of SEQ ID NO:4 or a biologically active variant thereof sharing at least 75% sequence identity therewith.
8. The method according to any one of the preceding items, wherein the second polypeptide is CfTPS3 of SEQ ID NO:4 or a biologically active variant thereof sharing at least 85% sequence identity therewith.
9. The method according to any one of items 1 to 6, wherein the second polypeptide is CfTPS4 of SEQ ID NO:3 or a biologically active variant thereof sharing at least 75% sequence identity therewith.
10. The method according to any one of items 1 to 6, wherein the second polypeptide is CfTPS4 of SEQ ID NO:3 or a biologically active variant thereof sharing at least 85% sequence identity therewith.
11. The method according to any one of the preceding items, wherein the first polypeptide has a sequence at least 70% identical to SEQ ID NO: 1 [CfTPS2] and the second polypeptide has a sequence at least 70% identical to SEQ ID NO: 3 [CfTPS4].
12. The method according to any one of the preceding items, wherein the first polypeptide is CfTPS2 of SEQ ID NO:1 or a biologically active variant thereof sharing at least 75% sequence identity therewith and the second polypeptide is CfTPS4 of SEQ ID NO:3 or a biologically active variant thereof sharing at least 75% sequence identity therewith.
13. The method according to any one of the preceding items, wherein the first polypeptide is CfTPS2 of SEQ ID NO:1 or a biologically active variant thereof sharing at least 85% sequence identity therewith and the second polypeptide is CfTPS4 of SEQ ID NO:3 or a biologically active variant thereof sharing at least 85% sequence identity therewith.
14. The method according to any one of items 1 to 6, wherein the first polypeptide has a sequence at least 70% identical to SEQ ID NO: 1 [CfTPS2] and the second polypeptide has a sequence at least 70% identical to SEQ ID NO: 4 [CfTPS3].
15. The method according to any one of items 1 to 6, wherein the first polypeptide is CfTPS2 of SEQ ID NO:1 or a biologically active variant thereof sharing at least 75% sequence identity therewith and the second polypeptide is CfTPS3 of SEQ ID NO:4 or a biologically active variant thereof sharing at least 75% sequence identity therewith.
16. The method according to any one of items 1 to 6, wherein the first polypeptide is CfTPS2 of SEQ ID NO:1 or a biologically active variant thereof sharing at least 85% sequence identity therewith and the second polypeptide is CfTPS3 of SEQ ID NO:4 or a biologically active variant thereof sharing at least 85% sequence identity therewith.
17. The method according to any one of the preceding items, wherein the first polypeptide has a sequence at least 70% identical to SEQ ID NO: 1 [CfTPS2] and the second polypeptide has a sequence at least 70% identical to SEQ ID NO: 5 [EpTPS8].
18. The method according to any one of items 1 to 6, wherein the first polypeptide is CfTPS2 of SEQ ID NO:1 or a biologically active variant thereof sharing at least 75% sequence identity therewith and the second polypeptide is EpTPS8 of SEQ ID NO:5 or a biologically active variant thereof sharing at least 75% sequence identity therewith.
19. The method according to any one of items 1 to 6, wherein the first polypeptide is CfTPS2 of SEQ ID NO:1 or a biologically active variant thereof sharing at least 85% sequence identity therewith and the second polypeptide is EpTPS8 of SEQ ID NO:5 or a biologically active variant thereof sharing at least 85% sequence identity therewith.
20. The method according to any one of the preceding items, wherein the first polypeptide has a sequence at least 70% identical to SEQ ID NO: 2 [SsLPPS] and the second polypeptide has a sequence at least 70% identical to SEQ ID NO: 3 [CfTPS4].
21. The method according to any one of the preceding items, wherein the first polypeptide is SsLPPS of SEQ ID NO:2 or a biologically active variant thereof sharing at least 75% sequence identity therewith and the second polypeptide is CfTPS4 of SEQ ID NO:3 or a biologically active variant thereof sharing at least 75% sequence identity therewith.
22. The method according to any one of the preceding items, wherein the first polypeptide is SsLPPS of SEQ ID NO:2 or a biologically active variant thereof sharing at least 85% sequence identity therewith and the second polypeptide is CfTPS4 of SEQ ID NO:3 or a biologically active variant thereof sharing at least 85% sequence identity therewith.
23. The method according to any one of the preceding items, wherein the first polypeptide has a sequence at least 70% identical to SEQ ID NO: 2 [SsLPPS] and the second polypeptide has a sequence at least 70% identical to SEQ ID NO: 4 [CfTPS3].
24. The method according to any one of the preceding items, wherein the first polypeptide is SsLPPS of SEQ ID NO:2 or a biologically active variant thereof sharing at least 75% sequence identity therewith and the second polypeptide is CfTPS3 of SEQ ID NO:4 or a biologically active variant thereof sharing at least 75% sequence identity therewith.
25. The method according to any one of the preceding items, wherein the first polypeptide is SsLPPS of SEQ ID NO:2 or a biologically active variant thereof sharing at least 85% sequence identity therewith and the second polypeptide is CfTPS3 of SEQ ID NO:4 or a biologically active variant thereof sharing at least 85% sequence identity therewith.
26. The method according to any one of the preceding items, wherein the first polypeptide has a sequence at least 70% identical to SEQ ID NO: 2 [SsLPPS] and the second polypeptide has a sequence at least 70% identical to SEQ ID NO: 5 [EpTPS8].
27. The method according to any one of the preceding items, wherein the first polypeptide is SsLPPS of SEQ ID NO:2 or a biologically active variant thereof sharing at least 75% sequence identity therewith and the second polypeptide is EpTPS8 of SEQ ID NO:5 or a biologically active variant thereof sharing at least 75% sequence identity therewith.
28. The method according to any one of the preceding items, wherein the first polypeptide is SsLPPS of SEQ ID NO:2 or a biologically active variant thereof sharing at least 85% sequence identity therewith and the second polypeptide is EpTPS8 of SEQ ID NO:5 or a biologically active variant thereof sharing at least 85% sequence identity therewith.
29. The method according to any one of the preceding items, wherein the first polypeptide and the second polypeptide are present at a stoichiometry ratio between 2:1 and 1:2, such as 1:1.
30. The method according to the preceding items, wherein the stoichiometry is 1:1.
31. The method according to any one of the preceding items, further comprising a step of recovering the (13R)-manoyl oxide.
32. The method according to any one of the preceding items, where the method is performed in vivo.
33. The method according to the preceding items, wherein the first and second polypeptides are heterologously expressed in a host organism.
34. The method according to the preceding items, wherein the host organism is a prokaryote or a eukaryote.
35. The method according to any one of the preceding items, wherein the host organism is selected from the group comprising bacteria, yeast, fungi, plants, insects and mammals.
36. The method according to any one of the preceding items, wherein the host organism is selected from the group comprising Escherichia coli, Saccharomyces cerevisiae, Schizosaccharomyces pombe, Nicotiana benthamiana and Physcomitrella patens.
37. The method according to any one of the preceding items, wherein the first and second polypeptides are purified after heterologous expression.
38. The method according to any one of the preceding items, wherein GGPP is provided in a composition.
39. The method according to any one of the preceding items, wherein the first polypeptide has a sequence at least 70% identical to, such as at least 75% identical to, such as at least 80% identical to, such as at least 85% identical to, such as at least 90% identical to, such as at least 95% identical to, such as 100% identical to SEQ ID NO: 1 [CfTPS2].
40. The method according to any one of items 1 to 38, wherein the first polypeptide has a sequence at least 70% identical to, such as at least 75% identical to, such as at least 80% identical to, such as at least 85% identical to, such as at least 90% identical to, such as at least 95% identical to, such as 100% identical to SEQ ID NO: 2 [SsLPPS].
41. The method according to any one of the preceding items, wherein the second polypeptide has a sequence at least 75% identical to, such as at least 80% identical to, such as at least 85% identical to, such as at least 90% identical to, such as at least 95% identical to, such as 100% identical to SEQ ID NO: 3 [CfTPS4].
42. The method according to any one of items 1 to 40, wherein the second polypeptide has a sequence at least 60% identical to, such as at least 65% identical to, such as at least 70% identical to, such as at least 75% identical to, such as at least 80% identical to, such as at least 85% identical to, such as at least 90% identical to, such as at least 95% identical to, such as 100% identical to SEQ ID NO: 4 [CfTPS3].
43. The method according to any one of items 1 to 40, wherein the second polypeptide has a sequence at least 60% identical to, such as at least 65% identical to, such as at least 70% identical to, such as at least 75% identical to, such as at least 80% identical to, such as at least 85% identical to, such as at least 90% identical to, such as at least 95% identical to, such as 100% identical to SEQ ID NO: 5 [EpTPS8].
44. The method according to any one of the preceding items, wherein:
   the first polypeptide has a sequence at least 70% identical to, such as at least 75% identical to, such as at least 80% identical to, such as at least 85% identical to, such as at least 90% identical to, such as at least 95% identical to, such as 100% identical to SEQ ID NO: 1 [CfTPS2]; and
   the second polypeptide has a sequence at least 75% identical to, such as at least 80% identical to, such as at least 85% identical to, such as at least 90% identical to, such as at least 95% identical to, such as 100% identical to SEQ ID NO: 3 [CfTPS4].
45. The method according to any one of the preceding items, wherein the method is performed in a host cell.
46. The method according to the preceding items, wherein the host cell is capable of expressing the first and second polypeptides in a stoichiometry of 1:1.
47. The method according to any one of the preceding items, wherein the host cell is selected from the group comprising bacterial cell, yeast cells, fungal cells, plant cells, mammalian cells and insect cells.
48. The method according to the preceding items, wherein the host cell is a bacterial cell.
49. The method according to any one of the preceding items wherein the host cell is Escherichia coli.
50. The method according to any one of the preceding items, wherein the host cell is a yeast cell.
51. The method according to any one of the preceding items, wherein the host cell is selected from the group consisting of Saccharomyces cerevisiae or Schizosaccharomyces pombe.
52. The method according to any one of items 45 to 51, wherein the host cell is capable of secreting (13R)-manoyl oxide.
53. The method according to any one of the preceding items, wherein the host cell is a plant cell.
54. The method according to any one of the preceding items, wherein the host cell is selected from the group consisting of Nicotiana benthamiana and Physcomitrella patens.
55. The method according to any one of the preceding items, wherein the host cell further is capable of producing GGPP.
56. The method according to any one of the preceding items, wherein GGPP is produced via the plastidial methylerythritol 4-phosphate (MEP) pathway.
57. The method according to any one of items 45 to 56, wherein the host cell comprises at least one heterologous nucleic acid encoding an enzyme of the MEP pathway.
58. The method according to any one of the preceding items, further comprising non-enzymatical synthesis of forskolin from (13R)-manoyl-oxide.
59. The method according to any one of the preceding items, further comprising the step of synthesising forskolin from (13R)-manoyl-oxide with the proviso that said step does not include enzymatic synthesis step.
60. A method of producing forskolin comprising the steps of
i) preparing (13R)-manoyl oxide by the method according to any one of items 1 to 57;
ii) synthesizing forskolin from said (13R)-manoyl oxide by non-enxymatical synthesis.
61. (13R)-manoyl oxide obtained by the method of any one of items 1 to 57.
65. The (13R)-manoyl oxide according to item 64, wherein said (13R)-manoyl oxide is more than 90% enantiomerically pure, such as more than 99% enantiomerically pure.
66. An isolated diterpene synthase (diTPS) polypeptide comprising:
i) an amino acid sequence selected from the group consisting of SEQ ID NO: 1 [CfTPS2], SEQ ID NO: 4 [CfTPS3], SEQ ID NO: 3 [CfTPS4] and SEQ ID NO: 5 [EpTPS8];
ii) a biologically active sequence variant of said polypeptide, wherein the sequence variant has at least 75% sequence identity to said SEQ ID NO: 1 [CfTPS2], SEQ ID NO: 4 [CfTPS3], SEQ ID NO: 3 [CfTPS4] or SEQ ID NO: 5 [EpTPS8]; or
iii) a biologically active fragment of at least 50 contiguous amino acids of any of i) through ii), said fragment having at least 75% sequence identity to SEQ ID NO: 1 [CfTPS2], SEQ ID NO: 4 [CfTPS3], SEQ ID NO: 3 [CfTPS4] or SEQ ID NO: 5 [EpTPS8] in a range of overlap of at least 50 amino acids,
wherein the biological activity is diterpene synthase activity.
67. The polypeptide according to item 66, wherein the polypeptide has a class I diTPS activity.
68. The polypeptide according to any one of items 66 to 67, wherein the sequence is at least 75% identical to, such as at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 4 [CfTPS3].
69. The polypeptide according to any one of items 66 to 67, wherein polypeptide is CfTPS3 of SEQ ID NO:4 or a biologically active variant thereof at least 80% identical to, such as at least 85% identical to, such as at least 90% identical to, such as at least 95% identical to, such as at least 96% identical to, such as at least 97% identical to, such as at least 98% identical to, such as at least 99% identical to SEQ ID NO: 4 [CfTPS3].
70. The polypeptide according to any one of the preceding items, wherein the sequence is at least 75% identical to, such as at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 3 [CfTPS4].
71. The polypeptide according to any one of items 66 to 67, wherein polypeptide is CfTPS4 of SEQ ID NO:3 or a biologically active variant thereof at least 80% identical to, such as at least 85% identical to, such as at least 90% identical to, such as at least 95% identical to, such as at least 96% identical to, such as at least 97% identical to, such as at least 98% identical to, such as at least 99% identical to SEQ ID NO: 3 [CfTPS4].
72. The polypeptide according to any one of the preceding items, wherein the sequence is at least 75% identical to, such as at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 5 [EpTPS8].
73. The polypeptide according to any one of items 66 to 67, wherein polypeptide is EpTPS8 of SEQ ID NO:5 or a biologically active variant thereof at least 80% identical to, such as at least 85% identical to, such as at least 90% identical to, such as at least 95% identical to, such as at least 96% identical to, such as at least 97% identical to, such as at least 98% identical to, such as at least 99% identical to SEQ ID NO: 5 [EpTPS8].
74. The polypeptide according to any one of items 66 to 73, comprising an operative class I DDxxD domain.
75. The polypeptide according to item 66, wherein the polypeptide has a class II diTPS activity.
76. The polypeptide according to item 75, having a sequence at least 75% identical to, such as at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 1 [CfTPS2].
77. The polypeptide according to any one of items 75 to 76, comprising an operative class II DxDD domain.
78. The polypeptide according to any one of items 65 to 82, further comprising a plastidial targeting signal.
79. A polynucleotide encoding a polypeptide as defined in any one of items 66 to 78.
80. The polynucleotide according to item 79, wherein the polynucleotide has a sequence with at least 85% identity to a sequence selected from the group consisting of SEQ ID NO:6 [CfTPS2], SEQ ID NO:9 [CfTPS3], SEQ ID NO:8 [CfTPS4] and SEQ ID NO:10 [EpTPS8].
81. The polynucleotide according to any one of items 79 to 80, wherein the polynucleotide has a sequence with at least 85% identity to SEQ ID NO: 6 [CfTPS2].
82. The polynucleotide according to any one of items 79 to 81, wherein the polynucleotide encodes CfTPS2 of SEQ ID NO:1 or a biologically active variant thereof at least 80% identical to, such as at least 85% identical to, such as at least 90% identical to, such as at least 95% identical to, such as at least 96% identical to, such as at least 97% identical to, such as at least 98% identical to, such as at least 99% identical to SEQ ID NO: 1 [CfTPS2].
83. The polynucleotide according to any one of items 79 to 80, wherein the polynucleotide has a sequence with at least 85% identity to SEQ ID NO: 9 [CfTPS3].
84. The polynucleotide according to any one of items 79, 80 and 83, wherein the polynucleotide encodes CfTPS3 of SEQ ID NO:4 or a biologically active variant thereof at least 80% identical to, such as at least 85% identical to, such as at least 90% identical to, such as at least 95% identical to, such as at least 96% identical to, such as at least 97% identical to, such as at least 98% identical to, such as at least 99% identical to SEQ ID NO: 4 [CfTPS3].
85. The polynucleotide according to any one of items 79 to 80, wherein the polynucleotide has a sequence with at least 85% identity to SEQ ID NO: 8 [CfTPS4].
86. The polynucleotide according to any one of items 79, 80 and 85, wherein the polynucleotide encodes CfTPS4 of SEQ ID NO:3 or a biologically active variant thereof at least 80% identical to, such as at least 85% identical to, such as at least 90% identical to, such as at least 95% identical to, such as at least 96% identical to, such as at least 97% identical to, such as at least 98% identical to, such as at least 99% identical to SEQ ID NO: 3 [CfTPS4].
87. The polynucleotide according to any one of items 79 to 80, wherein the polynucleotide has a sequence with at least 85% identity to SEQ ID NO: 10 [EpTPS8].
88. The polynucleotide according to any one of items 79, 80 and 87, wherein the polynucleotide encodes EpTPS8 of SEQ ID NO:5 or a biologically active variant thereof as at least 80% identical to, such as at least 85% identical to, such as at least 90% identical to, such as at least 95% identical to, such as at least 96% identical to, such as at least 97% identical to, such as at least 98% identical to, such as at least 99% identical to SEQ ID NO: 5 [EpTPS8].
89. The polynucleotide according to any one of items 79 to 88, further comprising a sequence coding for a plastidial targeting signal.
90. The polynucleotide according to any one of items 79 to 89, wherein the polynucleotide is codon-optimised for expression in a host cell.
91. The polynucleotide according to item 90, wherein the host cell is selected from the group comprising bacterial cell, yeast cells, fungal cells, plant cells, mammalian cells and insect cells.
92. A vector comprising at least one polynucleotide as defined in any one of items 79 to 91.
93. The vector according to item 92, wherein the vector comprises one polynucleotide as defined in any one of items 83 to 88 and one polynucleotide as defined in any one of items 81 to 82.
94. A host cell comprising the polynucleotide according to any one of items 79 to 91, and/or the vector according to any one of items 92 to 93.
95. The host cell according to item 94, capable of producing (13R)-manoyl oxide.
96. The cell according any one of items 94 to 95, wherein the cell expresses:
(i) a first polypeptide having a sequence at least 70% identical to SEQ ID NO: 1 [CfTPS2]; and
(ii) a second polypeptide having a sequence at least 70% identical to SEQ ID NO: 4 [CfTPS3], SEQ ID NO: 3 [CfTPS4], or SEQ ID NO: 5 [EpTPS8].
97. The cell according to any one of items 94 to 96, wherein the cell expresses:
   (i) A first polypeptide, which is the polypeptide according to any one of claims 75 to 77; and
   (ii) A second polypeptide, which is the polypeptide according to any one of claims 67 to 74.
98. The cell according to item 94, wherein the first polypeptide has a sequence at least 70% identical to SEQ ID NO: 1 [CfTPS2] and the second polypeptide has a sequence at least 70% identical to SEQ ID NO: 4 [CfTPS3].
99. The cell according to item 94, wherein the first polypeptide has a sequence at least 70% identical to SEQ ID NO: 1 [CfTPS2] and the second polypeptide has a sequence at least 70% identical to SEQ ID NO: 3 [CfTPS4].
100. The cell according to item 94, wherein the first polypeptide has a sequence at least 70% identical to SEQ ID NO: 1 [CfTPS2] and the second polypeptide has a sequence at least 70% identical to SEQ ID NO: 5 [EpTPS8].
101. The cell according to any one of items 94 to 100, wherein the cell is selected from the group comprising bacterial cell, yeast cells, fungal cells, plant cells, mammalian cells and insect cells.
102. The cell according to item 101, wherein the cell is a bacterial cell.
103. The cell according to item 102, wherein the bacteria is Escherichia coli.
104. The cell according to item 101, wherein the cell is a yeast cell.
105. The cell according to item 104, wherein the yeast is Saccharomyces cerevisiae or Schizosaccharomyces pombe.
106. The cell according to any one of items 94 to 105, wherein the cell is capable of secreting (13R)-manoyl oxide.
107. The cell according to any one of items 94 to 106, wherein the cell is transfected with at least one vector for expressing the first and the second polypeptides.
108. The cell according to any one of items 94 to 107, wherein the cell is transfected with at least one vector according to any one of items 92 to 93.
109. The cell according to item 101, wherein the cell is a plant cell selected from the group consisting of Nicotiana benthamiana and Physcomitrella patens.
110. The cell according to item 109, wherein the cell is a Nicotiana benthamiana cell.
111. The cell according to item 110, wherein the Nicotiana benthamiana cell is a leaf cell.
112. The cell according to item 111, wherein the Nicotiana benthamiana leaf cell is transfected with at least one vector for expressing the first and the second polypeptides.
113. The cell according to item 112, wherein the at least one vector for expressing the first and the second polypeptides is a bacterial vector for transient expression.
114. The cell according to item 113, wherein the bacterial vector is Agrobacterium tumefaciens.
115. The cell according to any one of items 113 to 114, wherein the first and second polypeptides are expressed in a transient manner.
116. The cell according to item 115, wherein the vector also encodes a suppressor of gene silencing.
117. The cell according to item 116, wherein the suppressor of gene silencing is the p19 protein of tomato bushy stunt virus.
118. The cell according to any one of items 94 to 117, wherein the cell is further capable of producing GGPP.
119. The cell according to item 118, wherein GGPP is produced via the plastidial methylerythritol 4-phosphate (MEP) pathway.
120. The cell according to any one of items 94 to 119, wherein the host cell comprises at least one heterologous nucleic acid encoding an enzyme of the MEP pathway.
121. The cell according to any one of items 94 to 120, wherein the cell is further engineered to direct metabolic fluxes.
122. The cell according to any one of items 94 to 121, wherein the (13R)-manoyl oxide is substantially pure.

1. A method of manufacturing (13R)-manoyl oxide, said method comprising the steps of:
(i) providing geranylgeranyl diphosphate (GGPP);
(ii) contacting GGPP of step (i) with a first polypeptide having a sequence at least 75% identical to SEQ ID NO: 1 [CfTPS2] or SEQ ID NO: 2 [SsLPPS], thus obtaining labd-13-en-8,15-diol diphosphate (LPP);
(iii) contacting the LPP of step (ii) with a second polypeptide having a sequence at least 75% identical to SEQ ID NO: 3 [CfTPS4], SEQ ID NO: 4 [CfTPS3] or SEQ ID NO: 5 [EpTPS8];
thus obtaining (13R)-manoyl-oxide.

2. The method according to any one of the preceding claims, wherein the (13R)-manoyl oxide obtained is enantiomerically pure.

3. The method according to any one of the preceding claims, wherein the first polypeptide and the second polypeptide are present at a stoichiometry ratio between 2:1 and 1:2, such as 1:1.

4. The method according to any one of the preceding claims, further comprising a step of recovering the (13R)-manoyl oxide.

5. The method according to any one of the preceding claims, where the method is performed in vivo.

6. The method according to the preceding claims, wherein the first and second polypeptides are heterologously expressed in a host organism selected from the group comprising bacteria, yeast, fungi, plants, insects and mammals.

7. The method according to any one of the preceding claims, wherein the host organism is selected from the group comprising Escherichia coli, Saccharomyces cerevisiae, Schizosaccharomyces pombe, Nicotiana benthamiana and Physcomitrella patens.

8. The method according to any one of the preceding claims, wherein the first and second polypeptides are purified after heterologous expression.

9. The method according to any one of the preceding claims, wherein GGPP is provided in a composition or is produced by the host organism.

10. The method according to any one of the preceding claims, wherein:
the first polypeptide has a sequence at least 75% identical to, such as at least 80% identical to, such as at least 85% identical to, such as at least 90% identical to, such as at least 95% identical to, such as 100% identical to SEQ ID NO: 1 [CfTPS2]; and
the second polypeptide has a sequence at least 75% identical to, such as at least 80% identical to, such as at least 85% identical to, such as at least 90% identical to, such as at least 95% identical to, such as 100% identical to SEQ ID NO: 3 [CfTPS4].

11. The method according to any one of the preceding claims, wherein the method is performed in a host cell selected from the group comprising bacterial cell, yeast cells, fungal cells, plant cells, mammalian cells and insect cells, wherein the host cell is capable of expressing the first and second polypeptides in a stoichiometry of 1:1.

12. The method according to any one of the preceding claims, further comprising non-enzymatical synthesis of forskolin from (13R)-manoyl-oxide.

13. A method for producing forskolin comprising the step of preparing (13R)-manoyl oxide by the method according to any one of the preceding claims, and the second step of synthesising forskolin from said (13R)-manoyl-oxide with the provisos that said second step does not include enzymatic synthesis steps.

14. (13R)-manoyl oxide obtained by the method of any one of claims 1 to 14.

15. The (13R)-manoyl oxide according to claim 15, wherein the (13R)-manoyl oxide is more than 90% enantiomerically pure.

16. An isolated diterpene synthase (diTPS) polypeptide comprising:
i) an amino acid sequence selected from the group consisting of SEQ ID NO: 1 [CfTPS2], SEQ ID NO:4 [CfTPS3], SEQ ID NO: 3 [CfTPS4] and SEQ ID NO:5 [EpTPS8];
ii) a biologically active sequence variant of said polypeptide, wherein the sequence variant has at least 80% sequence identity to said SEQ ID NO: 1 [CfTPS2], SEQ ID NO:4[CfTPS3], SEQ ID NO: 3 [CfTPS4] or SEQ ID NO:5 [EpTPS8],
wherein the biological activity is diterpene synthase activity.

17. The polypeptide according to claim 166, wherein the polypeptide has a class I diTPS activity, and has a sequence at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 4 [CfTPS3], SEQ ID NO: 3 [CfTPS4] or SEQ ID NO: 5 [EpTPS8].

18. The polypeptide according to claim 17, wherein the polypeptide is capable of catalysing cleavage of the diphosphate group of LPP and additional cyclization or rearrangement reactions on the resulting carbocation yielding (13R)-manoyl oxide.

19. The polypeptide according to claim 17, comprising an operative class I DDxxD domain.

20. The polypeptide according to claim 16, wherein the polypeptide has a class II diTPS activity, and has a sequence at least 80% identical to, such as at least 85% identical to, such as at least 90% identical to, such as at least 95% identical to, such as at least 96% identical to, such as at least 97% identical to, such as at least 98% identical to, such as at least 99% identical to, such as 100% identical to SEQ ID NO: 1 [CfTPS2].

21. The polypeptide according to claim 20, wherein the polypeptide is capable of catalysing cycloisomerisation of GGPP to LPP.

22. The polypeptide according to claim 20, comprising an operative class II DxDD domain.

23. The polypeptide according to any one of claims 16 to 22, further comprising a plastidial targeting signal.

24. A polynucleotide encoding a polypeptide as defined in any one of claims 16 to 23.

25. The polynucleotide according to claim 24, wherein the polynucleotide has a sequence with at least 85% identity to a sequence selected from the group consisting of SEQ ID NO:6 [CfTPS2], SEQ ID NO:9 [CfTPS3], SEQ ID NO:8 [CfTPS4], and SEQ ID NO: 10 [EpTPS8].

26. The polynucleotide according to any one of claims 24 to 25, further comprising a sequence coding for a plastidial targeting signal.

27. The polynucleotide according to any one of claims 24 to 26, wherein the polynucleotide is codon-optimised for expression in a host cell selected from the group comprising bacterial cell, yeast cells, fungal cells, plant cells, mammalian cells and insect cells.

28. A vector comprising at least one polynucleotide as defined in any one of claims 24 to 27.

29. A host cell comprising the polynucleotide according to any one of claims 24 to 27, and/or the vector according to claim 28.

30. The cell according to claim 29, wherein the cell expresses:
(i) a first polypeptide, which is CfTPS2 of SEQ ID NO:1 or a biologically active variant thereof at least 80% identical to SEQ ID NO: 1 [CfTPS2]; and
(ii) a second polypeptide which is CfTPS 3 of SEQ ID NO:4, CfTPS4 of SEQ ID NO:3, EpTPS8 of SEQ ID NO:5, or a biologically active variant thereof at least 80% identical to SEQ ID NO:4 [CfTPS3], SEQ ID NO:3 [CfTPS4] or SEQ ID NO:5 [EpTPS8],
wherein the cell is selected from the group comprising bacterial cell, yeast cells, fungal cells, plant cells, mammalian cells and insect cells.

31. The cell according to claim 30, wherein the cell is a Nicotiana benthamiana cell transfected with at least one viral vector for transiently expressing the first and the second polypeptides.

32. The cell according to any one of claims 29 to 31, wherein the cell is further naturally capable of producing or further engineered to produce GGPP via the plastidial methylerythritol 4-phosphate (MEP) pathway.


SEQUENCE LISTING (63 sequences)

SEQ ID NO: 1 - protein, 773 aa, Unknown
  TPS2 from Coleus forskohlii
MKMLMIKSQFRVHSIVSAWANNSNKRQSLGHQIRRKQRSQVTECRVASLDALNGIQKVGPATIGTPEEENKKIEDSIEYVKELLKTMGDGRISVSPYDTAIVALIKDLEGGDGPEFPSCLEWIAQNQLADGSWGDHFFCIYDRVVNTAACVVALKSWNVHADKIEKGAVYLKENVHKLKDGKIEHMPAGFEFVVPATLERAKALGIKGLPYDDPFIREIYSAKQTRLTKIPKGMIYESPTSLLYSLDGLEGLEWDKILKLQSADGSFITSVSSTAFVFMHTNDLKCHAFIKNALTNCNGGVPHTYPVDIFARLWAVDRLQRLGISRFFEPEIKYLMDHINNVWREKGVFSSRHSQFADIDDTSMGIRLLKMHGYNVNPNALEHFKQKDGKFTCYADQHIESPSPMYNLYRAAQLRFPGEEILQQALQFAYNFLHENLASNHFQEKWVISDHLIDEVRIGLKMPWYATLPRVEASYYLQHYGGSSDVWIGKTLYRMPEISNDTYKILAQLDFNKCQAQHQLEWMSMKEWYQSNNVKEFGISKKELLLAYFLAAATMFEPERTQERIMWAKTQVVSRMITSFLNKENTMSFDLKIALLTQPQHQINGSEMKNGLAQTLPAAFRQLLKEFDKYTRHQLRNTWNKWLMKLKQGDDNGGADAELLANTLNICAGHNEDILSHYEYTALSSLTNKICQRLSQIQDKKMLEIEEGSIKDKEMELEIQTLVKLVLQETSGGIDRNIKQTFLSVFKTFYYRAYHDAKTIDAHIFQVLFEPVV

SEQ ID NO: 2 - protein, 785 aa, Salvia sclarea
  SsLPPS (1)..(785)
MTSVNLSRAPAAITRRRLQLQPEFHAECSWLKSSSKHAPLTLSCQIRPKQLSQIAELRVTSLDASQASEKDISLVQTPHKVEVNEKIEESIEYVQNLLMTSGDGRISVSPYDTAVIALIKDLKGRDAPQFPSCLEWIAHHQLADGSWGDEFFCIYDRILNTLACVVALKSWNLHSDIIEKGVTYIKENVHKLKGANVEHRTAGFELVVPTFMQMATDLGIQDLPYDHPLIKEIADTKQQRLKEIPKDLVYQMPTNLLYSLEGLGDLEWERLLKLQSGNGSFLTSPSSTAAVLMHTKDEKCLKYIENALKNCDGGAPHTYPVDIFSRLWAIDRLQRLGISRFFQHEIKYFLDHIESVWEETGVFSGRYTKFSDIDDTSMGVRLLKMHGYDVDPNVLKHFKQQDGKFSCYIGQSVESASPMYNLYRAAQLRFPGEEVLEEATKFAFNFLQEMLVKDRLQERWVISDHLFDEIKLGLKMPWYATLPRVEAAYYLDHYAGSGDVWIGKSFYRMPEISNDTYKELAILDFNRCQTQHQLEWIHMQEWYDRCSLSEFGISKRELLRSYFLAAATIFEPERTQERLLWAKTRILSKMITSFVNISGTTLSLDYNFNGLDEIISSANEDQGLAGTLLATFHQLLDGFDIYTLHQLKHVWSQWFMKVQQGEGSGGEDAVLLANTLNICAGLNEDVLSNNEYTALSTLTNKICNRLAQIQDNKILQVVDGSIKDKELEQDMQALVKLVLQENGGAVDRNIRHTFLSVSKTFYYDAYHDDETTDLHIFKVLFRPVV

SEQ ID NO: 3 - protein, 587 aa, Unknown
  CfTPS4 from Coleus forskohlii
MSITINLRVIAFPGHGVQSRQGIFAVMEFPRNKNTFKSSFAVKCSLSTPTDLMGKIKEKLREKVDNSGAAMATDSADMPTNLCIVDSLQRLGVEKYFQSEIDTVLDDAYRLWQLKQKDIFSDITTHAMAFRLLRVKGYDVSSEELAPYADQEGMNLQTIDLAAVIELYRAAQERVAEEDSTLEKLYVWTSTFLKQQLLAGAIPDQKLHKQVEYYLKNYHGILDRMGVRKGLDLYDAGYYKALKAADRLVDLCNEDLLAFARQDFNINQAQHRKELEQLQRWYADCRLDKLEFGRDVVRVSNFLTSAILGDPELSEVRLVFAKHIVLVTRIDDFFDHGGPREESHKILELIKEWKEKPAGEYVSKEVEILYTAVYNTVNELAERANVEQGRNVEPFLRTLWVQILSIFKIELDTWSDDTALTLDDYLNNSWVSIGCRICILMSMQFIGMKLPEEMLLSEECVDLCRHVSMVDRLLNDVQTFEKERKENTGNAVSLLLAAHKGERAFSEEEAIAKAKYLADCNRRSLMQIVYKTGTIFPRKCKDMFLKVCRIGCYLYASGDEFTSPQQMMEDMKSLVYEPLQIHPPPAN

SEQ ID NO: 4 - protein, 598 aa, Unknown
  TPS3 from Coleus forskohlii
MSSLAGNLRVIPFSGNRVQTRTGILPVHQTPMITSKSSAAVKCSLTTPTDLMGKIKEVFNREVDTSPAAMTTHSTDIPSNLCIIDTLQRLGIDQYFQSEIDAVLHDTYRLWQLKKKDIFSDITTHAMAFRLLRVKGYEVASDELAPYADQERINLQTIDVPTVVELYRAAQERLTEEDSTLEKLYVWTSAFLKQQLLTDAIPDKKLHKQVEYYLKNYHGILDRMGVRRNLDLYDISHYKSLKAAHRFYNLSNEDILAFARQDFNISQAQHQKELQQLQRWYADCRLDTLKFGRDVVRIGNFLTSAMIGDPELSDLRLAFAKHIVLVTRIDDFFDHGGPKEESYEILELVKEWKEKPAGEYVSEEVEILFTAVYNTVNELAEMAHIEQGRSVKDLLVKLWVEILSVFRIELDTWTNDTALTLEEYLSQSWVSIGCRICILISMQFQGVKLSDEMLQSEECTDLCRYVSMVDRLLNDVQTFEKERKENTGNSVSLLQAAHKDERVINEEEACIKVKELAEYNRRKLMQIVYKTGTIFPRKCKDLFLKACRIGCYLYSSGDEFTSPQQMMEDMKSLVYEPLPISPPEANNASGEKMSCVSN

SEQ ID NO: 5 - protein, 792 aa, Euphorbia peplus
  EpTPS8 (1)..(792)
MQVSLSLTTGSEPCITRIHAPSDAPLKQRNNEREKGTLELNGKVSLKKMGEMLRTIENVPIVGSTSSYDTAWVGMVPCSSNSSKPLFPESLKWIMENQNPEGNWAVDHAHHPLLLKDSLSSTLACVLALHKWNLAPQLVHSGLDFIGSNLWAAMDFRQRSPLGFDVIFPGMIHQAIDLGINLPFNNSSIENMLTNPLLDIQSFEAGKTSHIAYFAEGLGSRLKDWEQLLQYQTSNGSLFNSPSTTAAAAIHLRDEKCLNYLHSLTKQFDNGAVPTLYPLDARTRISIIDSLEKFGIHSHFIQEMTILLDQIYSFWKEGNEEIFKDPGCCATAFRLLRKHGYDVSSDSLAEFEKKEIFYHSSAASAHEIDTKSILELFRASQMKILQNEPILDRIYDWTSIFLRDQLVKGLIENKSLYEEVNFALGHPFANLDRLEARSYIDNYDPYDVPLLKTSYRSSNIDNKDLWTIAFQDFNKCQALHRVELDYLEKWVKEYKLDTLKWARQKTEYALFTIGAILSEPEYADARISWSQNTVFVTIVDDFFDYGGSLDECRNLINLMHKWDDHLTVGFLSEKVEIVFYSMYGTLNDLAAKAEVRQGRCVRSHLVNLWIWVMENMLKEREWADYNLVPTFYEYVAAGHITIGLGPVLLIALYFMGYPLSEDVVQSQEYKGVYLNVSIIARLLNDRVTVKRESAQGKLNGVSLFVEHGRGAVDEETSMKEVERLVESHKRELLRLIVQKTEGSVVPQSCKDLAWRVSKVLHLLYMDDDGFTCPVKMLNATNAIVNEPLLLTS

SEQ ID NO: 6 - DNA, 2322 bp, Unknown
  CfTPS2 from Coleus forskohlii
atgaaaatgttgatgatcaaaagtcaatttcgtgtacattcaatagtcagtgcatgggcgaacaacagcaataaaaggcagtcattgggtcaccaaattcgtcgaaagcaaagatcacaagtaaccgagtgtcgagttgcaagtctggatgcgttgaatggaattcaaaaagtcggcccagccaccattgggactcctgaagaggaaaataaaaagattgaggattccattgagtacgtgaaggagttgttgaagacaatgggcgacgggcgaatcagcgtttccccgtacgacacagcaatagttgccctgattaaggacttggaaggaggtgatggaccagagtttccatcttgtctagagtggattgcacagaatcaactggctgatggttcttggggggatcacttcttctgtatttatgatcgggttgttaatacagcagcttgtgtggtcgccttaaagtcgtggaacgttcacgcagacaagattgagaaaggagcagtgtacctgaaggagaatgtgcataaacttaaagatgggaagattgagcacatgcccgcagggtttgaatttgtggttcctgccactcttgaaagagccaaagccttggggatcaaaggtcttccctatgatgatcctttcatcagggaaatttatagtgcaaaacaaacaagattgaccaagataccaaagggcatgatctacgaatctccaacttctttattatatagtttagacggtctggaaggcttggagtgggacaagatactgaaactgcagtcggccgatggctcattcatcacctctgtgtcgtctactgccttcgtattcatgcacaccaacgaccttaaatgccacgccttcatcaaaaatgccctcaccaattgcaacgggggagtaccccacacgtatccagtggatatcttcgcacgactttgggcagtggaccgactgcaacgcctcggaatatctcgattctttgagcctgagatcaaatatttaatggatcacatcaataacgtgtggagggagaagggagttttcagttcaaggcattcacaatttgcggatattgacgacacatccatgggcatcaggcttctgaaaatgcacggatacaatgtcaacccaaatgcacttgaacatttcaaacagaaagatgggaagtttacatgctatgctgatcaacatatcgagtctccatcccccatgtacaatctctacagggctgctcagcttcgttttccaggagaagaaattcttcaacaagcccttcaatttgcctataattttctacatgaaaacctagccagcaatcactttcaagaaaaatgggtcatatccgaccacctaattgatgaggtaaggatcgggctgaagatgccatggtacgccaccctaccgcgagtggaggcttcatactatcttcaacattatggtggatccagcgacgtatggattggcaaaactttatacagaatgccagaaatcagtaacgacacatacaaaatacttgcacaattggacttcaacaaatgtcaagcacaacatcagttggaatggatgtccatgaaagagtggtatcaaagtaataatgttaaagaatttgggataagcaagaaagaacttcttcttgcttactttttggctgctgcaaccatgtttgaacccgaacgcacacaagagaggattatgtgggcgaaaactcaagtcgtttctcggatgatcacatcatttctcaacaaagaaaacacaatgtcattcgacctaaagattgcacttttaacccaaccccaacatcaaataaatggttctgagatgaagaatggacttgctcaaactcttcctgcagccttccgacaactactcaaggaattcgacaaatacacaagacatcaattgaggaatacttggaacaaatggttgatgaaactgaagcaaggagatgacaatggcggcgcagatgcagagctccttgcaaacacattaaacatatgtgctggacataacgaagacatattatcgcactatgaatacaccgctctctcctccctcacaaacaaaatatgtcagcgtctaagtcaaattcaagataaaaagatgctggaaattgaggaggggagcataaaagataaggagatggagctcgaaatacaaacattggtgaagttagtcctccaagaaaccagtgggggtatcgatagaaacatcaagcaaacatttttatcagtattcaagacattttactacagggcctaccacgatgctaagactatcgatgcccatattttccaagtactatttgaaccagtggtctga

SEQ ID NO: 7 - DNA, 2358 bp, Salvia sclarea
  SsLPPS (1)..(2358)
atgacttctgtaaatttgagcagagcaccagcagcgattacccggcgcaggctgcagctacagccggaatttcatgccgagtgttcatggctgaaaagcagcagcaaacacgcgcccttgaccttgagttgccaaatccgtcctaagcaactctcccaaatagctgaattgagagtaacaagcctggatgcgtcgcaagcgagtgaaaaagacatttcccttgttcaaactccgcataaggttgaggttaatgaaaagatcgaggagtcaatcgagtacgtccaaaatctgttgatgacgtcgggcgacgggcgaataagcgtgtcaccctatgacacggcagtgatcgccctgatcaaggacttgaaagggcgcgacgccccgcagtttccgtcatgtctcgagtggatcgcgcaccaccaactggctgatggctcatggggcgacgaattcttctgtatttatgatcggattctaaatacattggcatgtgtcgtagccttgaaatcatggaaccttcactctgatattattgaaaaaggagtgacgtacatcaaggagaatgtgcataaacttaaaggtgcaaatgttgagcacaggacagcggggttcgaacttgtggttcctacttttatgcaaatggccacagatttgggcatccaagatctgccctatgatcatcccctcatcaaggagattgctgacacaaaacaacaaagattgaaagagatacccaaggatttggtttaccaaatgccaacgaatttactgtacagtttagaagggttaggagatttggagtgggaaaggctactgaaactgcagtcgggcaatggctccttcctcacttcgccgtcgtccaccgccgccgtcttgatgcataccaaagatgaaaaatgtttgaaatacatcgaaaacgccctcaagaattgcgacggaggagcaccacatacttatccagtcgatatcttctcaagactttgggcaatcgataggctacaacgcctaggaatttctcgtttcttccagcacgagatcaagtatttcttagatcacatcgaaagcgtttgggaggagaccggagttttcagtggaagatatacgaaatttagcgatattgatgacacgtccatgggcgttaggcttctcaaaatgcacggatacgacgtcgatccaaatgtactaaaacatttcaagcaacaagatggtaaattttcctgctacattggtcaatcggtcgagtctgcatctccaatgtacaatctttatagggctgctcaactaagatttccaggagaagaagttcttgaagaagccactaaatttgcctttaacttcttgcaagaaatgctagtcaaagatcgacttcaagaaagatgggtgatatccgaccacttatttgatgagataaagctggggttgaagatgccatggtacgccactctaccccgagtcgaggctgcatattatctagaccattatgctggttctggtgatgtatggattggcaagagtttctacaggatgccagaaatcagcaatgatacatacaaggagcttgcgatattggatttcaacagatgccaaacacaacatcagttggagtggatccacatgcaggaatggtacgacagatgcagccttagcgaattcgggataagcaaaagagagttgcttcgctcttactttctggccgcagcaaccatattcgaaccggagagaactcaagagaggcttctgtgggccaaaaccagaattctttctaagatgatcacttcatttgtcaacattagtggaacaacactatctttggactacaatttcaatggcctcgatgaaataattagtagtgccaatgaagatcaaggactggctgggactctgctggcaaccttccatcaacttctagacggattcgatatatacactctccatcaactcaaacatgtttggagccaatggttcatgaaagtgcagcaaggagagggaagcggcggggaagacgcggtgctcctagcgaacacgctcaacatctgcgccggcctcaacgaagacgtgttgtccaacaatgaatacacggctctgtccaccctcacaaataaaatctgcaatcgcctcgcccaaattcaagacaataagattctccaagttgtggatgggagcataaaggataaggagctagaacaggatatgcaggcgttggtgaagttagtgcttcaagaaaatggcggcgccgtagacagaaacatcagacacacgtttttgtcggtttccaagactttctactacgatgcctaccacgacgatgagacgaccgatcttcatatcttcaaagtactctttcgaccggttgtatga

SEQ ID NO: 8 - DNA, 1764 bp, Unknown
  CfTPS4 from Coleus forskohlii
atgtcaatcaccatcaaccttcgagttatcgctttccccggccatggagttcagagcaggcaaggaatatttgcagtcatggaatttccaaggaacaagaacacctttaaatcatcctttgctgttaaatgcagcctctctactccaacagatttgatgggaaagataaaagaaaagttgagcgagaaggttgataattctgtggcagccatggctactgattctgccgatatgcccactaatctctgcatcgtcgactccctccagaggctgggagtcgaaaaatatttccaatccgaaatcgacactgttctcgatgatgcataccggttatggcagctgaagcagaaagatatattttcagacattactactcatgcaatggcgtttagacttctgcgagtcaaaggatacgatgtttcatcagaggagctggctccatacgctgatcaagagggcatgaacttgcaaacgattgatctggcggcggtcatcgagctgtacagagcagcacaggagagagtggctgaggaagacagcactcttgagaaactgtatgtctggaccagcacctttctgaagcagcagttgctggctggcgccattcctgaccagaaattgcacaaacaggtggagtactacttgaagaactaccacggcatattagatagaatgggagttagaaaaggactcgacctgtatgatgctggctattacaaggccctcaaagctgcagataggttggttgatctatgcaatgaagaccttctagcatttgcaaggcaagattttaatattaaccaagcccaacaccgcaaagaacttgagcaactgcaaaggtggtatgcagattgtaggttggacaaactcgagtttggaagagatgtggtgcgtgtatcgaattttctgacttcagccatccttggtgatccagagctttctgaagtccgtctagtgtttgccaaacatattgtgctagtgactaggatagatgattttttcgatcatggcgggcctagagaagaatcacacaagatccttgaactaataaaagaatggaaagagaagccagctggagaatatgtttccaaagaagttgagatcctatataccgcggtgtacaatacggtaaacgagttggcagagagggcaaatgttgaacaagggcgaaatgttgaaccatttctacgtacactgtgggttcaaatactgtcgattttcaagatagagttggatacatggagcgatgacacagcactaaccttggatgattacttgaacaactcatgggtgtcgattggttgtagaatctgcattctcatgtccatgcaattcattggtatgaagttaccagaagaaatgcttctcagtgaagagtgcgttgatttgtgtaggcatgtttccatggtcgaccgtctgctcaatgatgtccaaacttttgagaaggaacggaaagaaaatacaggaaacgctgtgagccttctgctagcagctcacaagggtgaaagagccttcagtgaagaggaagccatagcaaaagcgaaatatttggctgactgtaacaggagaagtctgatgcagattgtgtataaaacaggaaccattttcccaagaaaatgcaaagatatgttcttgaaggtgtgcaggattggttgctatttgtatgcgagtggcgacgaatttacttcccctcaacaaatgatggaagatatgaagtcattagtttatgagcccctccaaattcaccctccacctgctaactaa

SEQ ID NO: 9 - DNA, 1704 bp, Unknown
  CfTPS3 from Coleus forskohlii
atgatcacctctaaatcatctgcagctgttaaatgcagcctcaccacgccaacagatttgatggggaaaataaaagaggtcttcaacagggaagtcgatacttctccggcagccatgactactcattctacagatataccctctaatctctgcataatcgacaccctccagaggctgggaatcgaccaatacttccaatccgaaatcgacgctgttctacatgatacatacaggttatggcaactgaaaaagaaagatatattttcggatattactactcatgcaatggcgttcagacttttgcgagtcaaaggatatgaagttgcatcagacgaactggctccatacgctgatcaagagcgcattaacctgcaaaccattgatgtgccgacagttgttgagctatacagagcagcacaggagagattaactgaagaagatagcactcttgagaaactgtatgtttggaccagcgcctttctgaagcagcagttgctcactgatgccattcctgacaagaaattgcacaaacaagtggaatactacttgaagaactaccatggcatattagatagaatgggagtgagacgaaacctcgacctatatgacataagccattataaaagtctcaaagctgctcacaggttctataatctgagtaatgaagatatcctagcatttgcgaggcaagattttaatattagccaagcccaacaccagaaagaacttcagcagctgcaaaggtggtatgcagattgtaggttggacacgttgaaatttggaagagatgtagtgcgtataggaaattttctgacttcagcaatgattggtgatcctgaattgtctgacctccgtctagcgtttgccaaacatatagtgctcgtaacacgtattgatgattttttcgatcacggtgggcctaaagaagaatcatacgagatccttgaattagtaaaagaatggaaagagaagccagcaggagaatatgtttctgaagaagttgaaatcctatttacagcagtatacaatacagtaaacgagttggcagaaatggctcatatcgaacaaggacgaagcgttaaagaccttctagttaaactgtgggttgaaatactatcagttttcagaatagaattggatacatggaccaacgacacagcacttaccttagaagagtacttgtcacaatcctgggtgtccattggctgcagaatctgcattctcatatcaatgcaattccaaggtgtaaaattatctgatgaaatgcttcagagtgaagaatgcactgatttgtgtcggtatgtttcaatggttgaccggctgctcaacgatgtgcaaacttttgagaaggaacgcaaggaaaatacaggaaatagtgtgagccttctgcaagcagctcacaaagatgaaagagtcattaatgaagaggaagcttgtataaaggtaaaagaattggctgaatataacaggagaaaactgatgcagattgtctacaaaacaggaaccattttcccaagaaaatgcaaagatctgtttttgaaggcatgcagaattggttgttatttgtactcaagtggcgacgaatttacttcgcctcaacaaatgatggaagatatgaagtcactggtttatgaacccctaccaatttctcctcctgaagctaataatgcaagtggagaaaaaatgagttgtgtcagcaactag

SEQ ID NO: 10 - DNA, 2379 bp, Euphorbia peplus
  EpTPS8 (1)..(2379)
atgcaagtctctctctccctcaccactggctccgagccttgcattaccagaatccatgctccatctgatgcaccattgaaacagcggaacaatgaaagagagaaggggacactagaactaaatgggaaagtttcgctgaagaaaatgggagagatgctgaggacaatagaaaatgtacctatagtaggtagtacgtcgtcgtatgatacagcatgggtcggtatggtgccttgttcatcgaattcgtcgaaaccgctatttccagaaagcttgaaatggataatggagaatcaaaatccagaagggaattgggcagttgatcatgctcatcatcctcttcttcttaaagactctctttcttccactcttgcttgtgtccttgctttgcacaaatggaatttggctccccagcttgttcactccggtttggacttcatcggctctaatctatgggcagctatggactttcgacaacgatctcctctcggatttgatgttatatttccaggaatgatccaccaagctattgatttgggcatcaatcttcctttcaacaactcttcaattgagaacatgctcactaacccacttttggacattcaaagttttgaagcaggaaaaactagccatattgcatactttgccgagggattaggaagtagattaaaggattgggaacaacttcttcaatatcaaacaagtaacggttcgctcttcaattcaccttctacaactgctgccgctgctattcatctacgtgacgaaaaatgtcttaattacttgcattctctaaccaaacaattcgataatggtgctgttccaacactttatcctctcgatgcgcgtaccagaatctccataatcgatagtttggaaaagtttgggatccattcacatttcatccaagaaatgacaattctactagatcaaatatacagcttctggaaagaagggaatgaagaaatatttaaagaccctggatgttgtgcaacagcattccgactgctgcgaaagcatggttatgatgtttcttcagattccttggcagagtttgagaaaaaagagatattttaccattcatcagcagctagtgcacacgaaatcgataccaagtctattctagaattattcagagcttcccaaatgaaaattttgcaaaatgaaccaatactcgacagaatttacgattggactagcatttttctgagagaccagctagtgaaaggtctaatcgaaaacaagagtctgtacgaagaagttaattttgctttgggacatccatttgctaatctggatagactcgaagctcgttcttacatcgacaattacgatccatatgatgtcccacttcttaagacatcttacaggtcatccaatattgataacaaagatctatggacaattgcattccaagatttcaacaagtgccaagccttgcaccgtgtggaacttgattatctggagaaatgggtgaaagaatacaaattggacactctgaagtgggcaaggcagaagactgagtatgcattatttacgataggcgcaatcctttcggagcctgaatacgctgatgctcgcatctcatggtcacagaatactgtttttgtgactattgttgatgatttctttgactatggtggttcgttggatgaatgtcgtaacctcattaaccttatgcacaagtgggatgatcacttaaccgttggattcttgtcggaaaaagtggaaatcgtattttattcgatgtatggcacactcaatgaccttgctgccaaagccgaagtacgacaaggccgatgtgttcgaagtcacttagttaatttatggatctgggtgatggaaaacatgttaaaggagagagaatgggcagattacaatctggtgcctacattttacgagtacgtagccgctggacatataactatcggcttaggacctgtgcttcttattgccctctattttatggggtatccgctttctgaggatgtggttcaaagtcaagaatacaagggtgtttatttgaatgtcagcatcattgctcgacttctaaatgatcgcgtaactgttaagagggaaagtgcgcaaggaaagcttaatggtgtgtcattgttcgtcgaacatggtcgtggcgcggttgatgaggaaactagtatgaaggaagtagaaagactggtagagagccataagagagaattattaagattgattgtgcagaaaacggaaggcagtgtcgtcccgcaaagttgcaaagatctagcttggagggttagcaaagttttgcaccttctatatatggatgatgatggttttacatgtcctgtgaagatgcttaatgctacaaatgcaattgtcaacgaaccactccttttaacttcataa

SEQ ID NO: 11 - protein, 4 aa, Unknown
  Class II diterpene synthase domain
DXDD

SEQ ID NO: 12 - protein, 5 aa, Unknown
  Class I diterpene synthase domain
DDXXD

SEQ ID NO: 13 - DNA, 28 bp, Artificial sequence
  Bri046 forward primer for amplification of CfTPS1
cagaatggggtctctatccactatgaac

SEQ ID NO: 14 - DNA, 22 bp, Artificial sequence
  Bri047 reverse primer for amplification of CfTPS1
cagcatattcaggcgactggtt

SEQ ID NO: 15 - DNA, 30 bp, Artificial sequence
  Bri048 forward primer for amplification of CfTPS2
agattgaggattccattgagtacgtgaagg

SEQ ID NO: 16 - DNA, 30 bp, Artificial sequence
  Bri049 reverse primer for amplification of CfTPS2
gaagtttaatatccttcattctttattaca

SEQ ID NO: 17 - DNA, 27 bp, Artificial sequence
  Bri099 forward primer for amplification of CfTPS3
agctccattcaactagagtcatgtcgt

SEQ ID NO: 18 - DNA, 28 bp, Artificial sequence
  Bri100 reverse primer for amplification of CfTPS3
ttcatctggcttaactagttgctgacac

SEQ ID NO: 19 - DNA, 25 bp, Artificial sequence
  Bri0101 forward primer for amplification of CfTPS4
gtgcactctccaccaacgataaact

SEQ ID NO: 20 - DNA, 25 bp, Artificial sequence
  Bri102 reverse primer for amplification of CfTPS4
gcttcacagcctatgaatacatgat

SEQ ID NO: 21 - DNA, 22 bp, Artificial sequence
  Bri051 forward primer for amplification of CfTPS14
tatgacacggcatgggttgcta

SEQ ID NO: 22 - DNA, 29 bp, Artificial sequence
  Bri052 reverse primer for amplification of CfTPS14
tcactcaaaatttattctaagacaagagg

SEQ ID NO: 23 - DNA, 26 bp, Artificial sequence
  Bri064 forward primer for E. coli expression of CfTPS1
gctttagcaacatgtcatggatgaac

SEQ ID NO: 24 - DNA, 22 bp, Artificial sequence
  Bri065 reverse primer for E. coli expression of CfTPS1
cagcactcgagggcgactggtt

SEQ ID NO: 25 - DNA, 25 bp, Artificial sequence
  Bri068 forward primer for E. coli expression of CfTPS2
cacaagtaatcatgagtcgagttgc

SEQ ID NO: 26 - DNA, 23 bp, Artificial sequence
  Bri069 reverse primer for E. coli expression of CfTPS2
ccaatgttctcgagcactggttc

SEQ ID NO: 27 - DNA, 37 bp, Artificial sequence
  Bri146 forward primer for E. coli expression of CfTPS3
aggagatataccatggctccgatgatcacctctaaat

SEQ ID NO: 28 - DNA, 35 bp, Artificial sequence
  Bri147 reverse primer for E. coli expression of CfTPS3
ggtggtggtgctcgaggttgctgacacaactcatt

SEQ ID NO: 29 - DNA, 26 bp, Artificial sequence
  Bri117 forward primer for E. coli expression of CfTPS4
catcctttgtcatgaaatgcagcctc

SEQ ID NO: 30 - DNA, 29 bp, Artificial sequence
  Bri118 reverse primer for E. coli expression of CfTPS4
ttgttaggcggccgctggagggtgaattt

SEQ ID NO: 31 - DNA, 25 bp, Artificial sequence
  Bri066 forward primer for E. coli expression of CfTPS14
ttacgccatggcttccctggaagtt

SEQ ID NO: 32 - DNA, 27 bp, Artificial sequence
  Bri 067 reverse primer for E. coli expression of CfTPS14
actcaactcgagttctaagacaagagg

SEQ ID NO: 33 - DNA, 33 bp, Artificial sequence
  oSSB156 forward primer for tobacco expression of CfTPS1
ggcttaauatggggtctctatccactatgaacc

SEQ ID NO: 34 - DNA, 26 bp, Artificial sequence
  oSSB157 reverse primer for tobacco expression of CfTPS1
ggtttaautcaggcgactggttcgaa

SEQ ID NO: 35 - DNA, 34 bp, Artificial sequence
  oSSB158 forward primer for tobacco expression of CfTPS2
ggcttaauatgaaaatgttgatgatcaaaagtca

SEQ ID NO: 36 - DNA, 35 bp, Artificial sequence
  oSSB159 reverse primer for tobacco expression of CfTPS2
ggtttaautcagaccactggttcaaatagtacttg

SEQ ID NO: 37 - DNA, 24 bp, Artificial sequence
  oSSB178 forward primer for tobacco expression of CfTPS3
ggcttaauatgtcgtccctcgccg

SEQ ID NO: 38 - DNA, 35 bp, Artificial sequence
  oSSB179 reverse primer for tobacco expression of CfTPS3
ggtttaauctagttgctgacacaactcattttttc

SEQ ID NO: 39 - DNA, 31 bp, Artificial sequence
  oSSB180 forward primer for tobacco expression of CfTPS4
ggcttaauatgtcaatcaccatcaaccttcg

SEQ ID NO: 40 - DNA, 32 bp, Artificial sequence
  oSSB181 reverse primer for tobacco expression of CfTPS4
ggtttaauttagttagcaggtggagggtgaat

SEQ ID NO: 41 - DNA, 31 bp, Artificial sequence
  oSSB160 forward primer for tobacco expression of CfTPS14
ggcttaauatgtctctcccgctctctacttg

SEQ ID NO: 42 - DNA, 41 bp, Artificial sequence
  oSSB161 reverse primer for tobacco expression of CfTPS14
ggtttaauttattctaagacaagaggttgataaataattgc

SEQ ID NO: 43 - DNA, 22 bp, Artificial sequence
  CfTPS1 forward primer for qPCR
tagtctggaagggctggagaat

SEQ ID NO: 44 - DNA, 24 bp, Artificial sequence
  CfTPS1 reverse primer for qPCR
ttggtagcatttaggatcacgagt

SEQ ID NO: 45 - DNA, 28 bp, Artificial sequence
  CfTPS2 forward primer for qPCR
gacatattatcgcactatgaatacaccg

SEQ ID NO: 46 - DNA, 29 bp, Artificial sequence
  CfTPS2 reverse primer for qPCR
ctaacttcaccaatgtttgtatttcgagc

SEQ ID NO: 47 - DNA, 29 bp, Artificial sequence
  CfTPS3 forward primer for qPCR
catgcagaattggttgttatttgtactca

SEQ ID NO: 48 - DNA, 27 bp, Artificial sequence
  CfTPS3 reverse primer for qPCR
tttctccacttgcattattagcttcag

SEQ ID NO: 49 - DNA, 25 bp, Artificial sequence
  CfTPS4 forward primer for qPCR
aacggaaagaaaatacaggaaacgc

SEQ ID NO: 50 - DNA, 26 bp, Artificial sequence
  CfTPS4 reverse primer for qPCR
cagccaaatatttcgcttttgctatg

SEQ ID NO: 51 - DNA, 27 bp, Artificial sequence
  CfTPS14 forward primer for qPCR
atgcatatgtatcatttgctctagggc

SEQ ID NO: 52 - DNA, 30 bp, Artificial sequence
  CfTPS14 reverse primer for qPCR
ggatttgaatagattgtggtagtcagcatg

SEQ ID NO: 53 - DNA, 20 bp, Artificial sequence
  CfEF1a forward primer for qPCR reference
tgcatcacgaggctctccag

SEQ ID NO: 54 - DNA, 20 bp, Artificial sequence
  CfEF1a reverse primer for qPCR reference
ggcaacaaacccacgcttca

SEQ ID NO: 55 - DNA, 23 bp, Artificial sequence
  CfH3 forward primer for qPCR reference
gagattcgcaagtaccagaagag

SEQ ID NO: 56 - DNA, 23 bp, Artificial sequence
  CfH3 reverse primer for qPCR reference
aatcgcagatcagtcttgaagtc

SEQ ID NO: 57 - DNA, 21 bp, Artificial sequence
  CfTIF4a forward primer for qPCR reference
ctatgatctgccaactcagcc

SEQ ID NO: 58 - DNA, 21 bp, Artificial sequence
  CfTIF4a reverse primer for qPCR reference
ccttggtcacgaagtttatgg

SEQ ID NO: 59 - DNA, 23 bp, Artificial sequence
  gCfTIF4a forward primer for qPCR gDNA check
tttgacatgytgagaaggcagtc

SEQ ID NO: 60 - DNA, 21 bp, Artificial sequence
  gCfTIF4a reverse primer for qPCR gDNA check
aacatagaactgcttgatacc

SEQ ID NO: 61 - DNA, 23 bp, Artificial sequence
  gCfEF1a forward primer for qPCR gDNA check
tactactgcactgtcattgatgc

SEQ ID NO: 62 - DNA, 22 bp, Artificial sequence
  gCfEF1a reverse primer for qPCR gDNA check
tggacctctcaatcatgttgtc

SEQ ID NO: 63 - DNA, 1710 bp, Unknown
  CfTPS3 codon optimised for E. coli
atgggcattaccagcaagtcctccgcagcagtcaaatgtagcctgaccacgccgaccgacctgatgggcaagattaaagaagttttcaatcgtgaagtggataccagcccggcagcaatgaccacgcacagtacggacatcccgtccaacctgtgcattatcgataccctgcaacgtctgggcatcgaccaatattttcagtctgaaattgatgcagttctgcatgacacctaccgcctgtggcagctgaaaaagaaagatatctttagtgacatcaccacgcacgcgatggccttccgtctgctgcgtgtcaaaggttatgaagtggcgagcgatgaactggcaccgtacgctgaccaagaacgtatcaatctgcagaccattgatgtcccgacggtggttgaactgtatcgtgcagctcaggaacgcctgaccgaagaagatagtacgctggaaaagctgtacgtttggaccagcgcgtttctgaaacagcaactgctgacggatgccattccggacaagaaactgcataagcaggttgaatattacctgaaaaactatcacggcatcctggatcgcatgggtgtccgtcgcaatctggatctgtatgacattagccattacaagtctctgaaagcggcccaccgtttctacaacctgtcaaatgaagatatcctggcatttgctcgccaagacttcaacatttcgcaggcacaacatcagaaagaactgcagcaactgcagcgttggtatgctgattgtcgcctggacaccctgaaatttggccgtgatgtcgtgcgcatcggtaatttcctgacgagcgccatgattggcgatccggaactgtctgacctgcgtctggcgtttgccaaacatatcgttctggtcacccgcattgatgactttttcgatcacggcggtccgaaggaagaaagctatgaaatcctggaactggtgaaagaatggaaggaaaaaccggcgggcgaatatgtctctgaagaagtggaaatcctgtttaccgccgtttacaacacggtcaatgaactggcggaaatggcccacattgaacagggtcgtagtgtgaaggatctgctggtgaaactgtgggttgaaatcctgtccgttttccgcattgaactggatacctggacgaacgacaccgcactgacgctggaagaatatctgagtcagtcctgggtgtcaattggctgccgcatttgtatcctgatttcgatgcaatttcagggtgttaaactgtcagatgaaatgctgcagtcggaagaatgcaccgacctgtgtcgttacgtgagcatggttgatcgcctgctgaatgacgtccaaaccttcgaaaaggaacgtaaagaaaacacgggcaattcagtgtcgctgctgcaggcagctcataaagatgaacgcgttatcaacgaagaagaagcgtgcattaaggtcaaagaactggctgaatataatcgtcgcaagctgatgcagatcgtgtacaaaaccggtacgatttttccgcgtaagtgtaaagacctgtttctgaaagcgtgccgcattggctgttatctgtacagctctggtgatgaatttaccagcccgcagcaaatgatggaagacatgaaatctctggtttatgaaccgctgccgattagcccgccggaagccaacaatgcgtcaggtgaaaagatgtcgtgtgtctcaaacctcgag